(19) United States
(12) Patent Application Publication
(10) Pub. No.: US 2007/0275363 A1
(43) Pub. Date: Nov. 29, 2007

(54) Bioreactor for organ reconstruction and augmentation

(76) Inventors: Timothy A. Bertram, Winston-Salem, NC (US); Andrew Bruce, Lexington, NC (US); Deepak Jain, Winston-Salem, NC (US); John Ludlow, Carrboro, NC (US); Darrell McCoy, Clemmon, NC (US); Namrata Sangha, Winston-Salem, NC (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/706,173

(22) Filed: Feb. 12, 2007

Related U.S. Application Data
(60) Provisional application No. 60/772,800, filed on Feb. 10, 2006.

(51) Int. Cl. A01N 1/00; C12M 1/12

(52) U.S. Cl. 435/1.2; 435/297.3

(57) ABSTRACT

Bioreactors are used in neo-organ production to allow for an appropriate environment for the maintenance of healthy culturing conditions from pre-wetting to shipment of the neo-organ. The closed system “all-in-one bioreactor” is designed to allow for minimal exposure of the scaffold to the open air in order to maintain sterility. The design allows for the same container to be utilized for sterilization, pre-wetting, cell seeding, medium exchange, and shipment. The “all-in-one” bioreactor also remains completely closed after the urothelial cell seeding step to the implantation at the clinical site. This allows for sufficient time for release testing to occur so the neo-organ can be implanted into the patient.

### RELATED APPLICATIONS

This application claims the benefit of U.S. Provisional Application No. 60/772,800, filed Feb. 10, 2006, the contents of which are hereby incorporated by reference in their entirety.

### FIELD OF THE INVENTION

The invention is directed to methods and materials for tissue reconstruction, repair, augmentation and replacement, and particularly to use of such treatments in patients having a defect in urogenital tissues such as the bladder. The invention is also directed to a closed system bioreactor and methods and materials for using this closed system bioreactor for neo-organ sterilization, pre-wetting, seeding, medium exchange, and shipping.

### BACKGROUND OF THE INVENTION

The medical community has directed considerable attention and effort to the substitution of defective organs with operationally effective replacements. The replacements have ranged from completely synthetic constructs such as artificial hearts to completely natural organs from another mammalian donor. The field of heart transplants has been especially successful with the use of both synthetic hearts and natural hearts from living donors. Equal success has not been achieved in many other organ fields particularly in the field of bladder reconstruction.

The human urinary bladder is a musculomembranous sac, situated in the anterior part of the pelvic cavity, that serves as a reservoir for urine, which it receives through the ureters and discharges through the urethra. In a human the bladder is found in the pelvis behind the pelvic bone (pubic symphysis) and is above and posterior to a drainage tube, called the urethra, that exits to the outside of the body. The bladder, ureters, and urethra are all similarly structured in that they comprise muscular structures lined with a membrane comprising urothelial cells coated with mucus that is impermeable to the normal soluble substances of the urine. The trigone of the bladder, also called the trigonum vesicae, is a smooth triangular portion of the mucous membrane at the base of the bladder. The bladder tissue is elastic and compliant. That is, the bladder changes shape and size according to the amount of urine it contains. A bladder resembles a deflated balloon when empty but becomes somewhat pear-shaped and rises into the abdominal cavity when the amount of urine in it increases.

The bladder wall has three main layers of tissues: the mucosa, submucosa, and detrusor. The mucosa, comprising urothelial cells, is the innermost layer and is composed of transitional cell epithelium. The submucosa lies immediately beneath the mucosa and its basement membrane. It is composed of blood vessels which supply the mucosa with nutrients and the lymph nodes which aid in the removal of waste products. The detrusor is a layer of smooth muscle cells which expands to store urine and contracts to expel urine.

The urinary bladder is subject to numerous maladies and injuries which cause deterioration of the urinary bladder in patients. For example, bladder deterioration may result from infectious diseases, neoplasms and developmental abnormalities. Further, bladder deterioration may also occur as a result of trauma such as, for example, car accidents and sports injury.

Although a large number of bio-materials, including synthetic and naturally-derived polymers, have been employed for tissue reconstruction or augmentation (see, e.g., “Textbook of Tissue Engineering” Eds. Lanza, R., Langer, R., and Chick, W, ACM Press, Colorado (1996) and references cited therein), many materials have proven to be unsatisfactory for use in bladder reconstruction. For example, synthetic biomaterials such as polyvinyl and gelatin sponges, polytetrafluoroethylene (Teflon) felt, and silastic patches have been relatively unsuccessful, generally due to foreign body reactions (see, e.g., Kudish, H. G., J. Urol. 78:232 (1957); Ashkar, L. and Heller, E., J. Urol. 98:91 (1967); Kelami, A. et al., J. Urol. 104:693 (1970)). Other attempts have usually failed due to either mechanical, structural, functional, or biocompatibility problems. Permanent synthetic materials have been associated with mechanical failure and calculus formation.

Naturally-derived materials such as lyophilized dura, deepithelialized bowel segments, and small intestinal submucosa (SIS) have also been proposed for bladder replacement (for a general review, see Mooney, D. et al., “Tissue Engineering: Urogenital System” in “Textbook of Tissue Engineering” Eds. Lanza, R., Langer, R., and Chick, W., ACM Press, Colorado (1996)). However, it has been reported that bladders augmented with dura, peritoneum, placenta and fascia contract over time (Kelami, A. et al., J. Urol. 105:518 (1971)). De-epithelized bowel segments demonstrated an adequate urothelial covering for use in bladder reconstruction, but difficulties remain with either mucosal regrowth, segment fibrosis, or both. It has been shown that de-epithelization of the intestinal segments may lead to mucosal regrowth, whereas removal of the mucosa and submucosa may lead to retraction of the intestinal segment (see, e.g., Atala, A., J. Urol. 156:338 (1996)).

Other problems have been reported with the use of certain gastrointestinal segments for bladder surgery including stone formation, increased mucus production, neoplasia, infection, metabolic disturbances, long term contracture and resorption. These attempts with natural or synthetic materials have shown that bladder tissue, with its specific muscular elastic properties and urothelial impermeability functions, cannot be easily replaced.

Due to the multiple complications associated with the use of gastrointestinal segments for bladder reconstruction, investigators have sought alternate solutions. Recent surgical approaches have relied on native urological tissue for reconstruction, including auto-augmentation and ureterocystoplasty. However, auto-augmentation has been associated with disappointing long-term results and ureterocystoplasty is limited to cases in which a dilated ureter is already present. A system of progressive dilation for ureters and bladders has been proposed, however, this has not yet been attempted clinically. Sero-muscular grafts and de-epithelialized bowel segments, either alone or over a native urothelium, have also been attempted. However, graft shrinkage and re-epithelialization of initially de-epithelialized bowel segments has been a recurring problem.

One significant limitation besetting bladder reconstruction is directly related to the availability of donor tissue. The limited availability of bladder tissue prohibits the frequent routine reconstruction of bladder using normal bladder tissue. The bladder tissue that is available, and considered usable, may itself include inherent imperfections and disease. For example, in a patient suffering from bladder cancer, the remaining bladder tissue may be contaminated with metastasis. Accordingly, the patient is predestined to less than perfect bladder function.

Accordingly, there exists a need for methods and constructs for the reconstruction, repair, augmentation or replacement of organs or tissue structures in a patient in need of such treatment. In addition, there is a need for artificial organ constructs with improved biomechanical properties. Along with this challenge arises the need to design and implement a bioreactor that allows for as little manipulation as possible of the neo-organ from the step of sterilizing the unseeded scaffold to the shipping step in order to minimize the risk of handling error and meet the release criteria to ensure delivery of a safe product. Thus, there exists a need for a system capable of producing such artificial organ constructs, particularly for sterilizing, pre-wetting, seeding, medium exchange, and shipping of these neo-organ constructs.

### BRIEF SUMMARY OF THE INVENTION

Biocompatible synthetic or natural scaffolds are provided for the reconstruction, repair, augmentation or replacement of organs or tissue structures in a patient in need of such treatment.

The scaffolds are shaped to conform to at least a part of the organ or tissue structure and may be seeded with one or more cell populations. The seeded scaffolds are implanted into the patient at the site in need of treatment to form an organized organ or tissue structure. The scaffolds may be used to form organs or tissues, such as bladders, urethras, valves, and blood vessels.

The methods described herein for the reconstruction, repair, augmentation or replacement of laminarily organized luminal organs or tissue structures in a patient in need of such treatment includes the steps of providing at least a first population of cells, wherein the cells are cultured in a medium containing a suitable antibiotic; providing a biocompatible synthetic or natural polymeric matrix shaped to conform to at least a part of the luminal organ or tissue structure in need of the treatment; depositing the first cell population on or in a first area of the polymeric matrix, the first cell population being substantially a muscle cell population; depositing a second cell population of a different cell type than the first cell population in a second area of the polymeric matrix, the second area being substantially separated from the first area; and implanting the shaped polymeric matrix cell construct into the patient at the site of the treatment for the formation of laminarily organized luminal organ or tissue structure. For example, in a preferred embodiment, the laminarily organized luminal organ or tissue structure is formed in vivo, i.e., after the cell-seeded matrix construct is implanted into the patient at the site of treatment. In this embodiment, the laminar organization of the cells occurs post-implantation.

The biocompatible material is, for example, biodegradable. In some preferred embodiments, the biocompatible material is polyglycolic acid. In some preferred embodiments, the second cell population is substantially a urothelial cell population, and the first cell population is, for example, a smooth muscle cell population.

Suitable antibiotics for use in the constructs and methods described herein include any antibiotic that does not inhibit or impede cell growth. For example, the antibiotic does not inhibit the cell growth of first cell population such as a smooth muscle cell population. Alternatively or in addition, the antibiotic does not inhibit the cell growth of a second cell population such as a urothelial cell population. Preferably, the antibiotic is selected from gentamicin and vancomycin, and more preferably, the antibiotic is gentamicin.

These methods are used to treat, repair, replace or augment a luminal organ or tissue structure such as, for example, a genitourinary organ. The luminal organ or tissue structure is, e.g., a bladder, ureters or urethra. For example, the luminal organ or tissue structure is a bladder or bladder segment that has urothelial cells deposited on the inner surface of the matrix and smooth muscle cells deposited on the outer surface of the matrix. In one embodiment, the first and second cell populations are deposited sequentially. Alternatively, the first and second cell populations are deposited on separate matrix layers and the matrix layers are combined after the deposition steps. Upon implantation, wherein the laminarily organized luminal organ or tissue structure formed in vivo exhibits the compliance and/or urodynamic profile of natural bladder tissue.

Biocompatible synthetic or natural scaffolds are provided for the reconstruction, repair, augmentation or replacement of organs or tissue structures in a patient in need of such treatment. Sterility must be maintained throughout all procedures in the creation of neo-organ constructs. In particular, sterility must be maintained at the end of the process when the scaffolds undergo pre-wetting, seeding and shipping to the clinical site. It is vital to obtain the results of release testing in a timely manner. By implementing an “all-in-one” bioreactor that remains closed for the last several days of the neo-organ production process, release testing can be completed before the neo-organ is implanted.

The closed system “all-in-one” bioreactor consists of a single container for the neo-organ construct from the sterilization step to the shipping step in the process and is a closed system from the cell seeding step on to shipping. This accomplishes the goal of allowing three days for product release testing since the bioreactor is not physically opened after the cells are seeded until the time at which the surgeon opens the container to remove the neo-organ for implantation.

### BRIEF DESCRIPTION OF THE DRAWINGS

FIG. 1 is an illustration depicting a template for a multi-petal-shaped neo-organ matrix or scaffold. The edges of the petals are mated to form a quasi-spherical shaped hollow matrix.

FIGS. 2 and 3 are illustrations depicting the initial seeding vessel and bioreactor for use in seeding and growing neo-organ tissue scaffolds. Note that the bioreactor must be opened completely to seed and change medium.

FIG. 4 is an illustration the presence of smooth muscle cells on and in the polymeric matrix of a neobladder scaffold.

FIG. 5 is an illustration depicting the presence of urothelial cells on and in the polymeric matrix of a neobladder scaffold.

FIGS. 6-9 are illustrations depicting containers for packing and shipping cell-seeded neo-organ scaffolds. Note that the neo-bladder must be removed from the seeding bioreactor and manipulated with hemostats and forceps for attachment to the inner basket of the shipping container.

FIG. 6 depicts a shipping container with a screw-cap lid for packing and transporting cell-seeded neo-organ constructs.

FIG. 7 depicts an aerial view of the shipping container depicted in FIG. 6, without the screw-cap lid, showing an inner basket supporting a cell-seeded neo-organ construct.

FIG. 8 depicts the inner support basket shown in FIG. 7 with a cell-seeded neo-organ construct inside the basket.

FIG. 9 depicts a temperature controlled, insulated box used to ship the neo-organ construct shipping container depicted in FIG. 6.

FIGS. 10-12 are illustrations depicting the original concept for the design of a novel closed system “all-in-one” neo-organ bioreactor system.

FIGS. 13-16 are digital images of the basic and essential ideas for the first prototype for the “all-in-one” closed neo-bladder bioreactor system. This prototype was built to demonstrate the gyroscope movement of the rings used to seed both sides of the scaffold.

FIGS. 17-24 are illustrations depicting components for the first prototype for the “all-in-one” bioreactor. FIG. 17 shows all ring assemblies that will accommodate 150, 250, 350, and 450 mL scaffolds as well as the o-ring to seal the lid to the container. FIG. 18 shows the outer container and lid with seeding port and slots for filter material. FIG. 19 shows the fittings used to attach tubing for culture medium filling and removing, as well as the flared down tubes to allow filling from the bottom up to eliminate splashing. FIG. 20 is a close up of the flared tubing and tubing for the medium in and out ports. FIG. 21 shows the shipping lid with an o-ring that will enclose the medium ports and sealed-off tubing to protect them during the shipping process. FIG. 22 is another view of the shipping lid in place. FIG. 23 illustrates the bioreactor container with shipping lid in place. FIG. 24 depicts another angle of the bioreactor with its shipping lid attached.

FIGS. 25-26 are digital images of the rapid prototype for the initial “all-in-one” bioreactor design. FIG. 25 shows all components of the rapid prototype including the outer container, ring assembly, lid (with seeding port, culture medium in and out ports, down tubes, and slots for filter paper), seeding port lid, and shipping lid. FIG. 26 is a close-up of the main lid. FIG. 27 is a close up of the gyroscope ring assembly holding a scaffold. FIG. 28 illustrates how the ring assembly snaps into place at a notch in the main container.

FIGS. 29-33 are illustrations depicting the final design for the first prototype after changes were implemented based upon handling of the rapid prototype. FIG. 29 illustrates the presence of O-rings where the seeding lid and the shipping lid will be attached. FIG. 30 shows the new lid design that shows a larger seeding port and 2 additional down ports added for use in gas exchange. Note that the slots for filter paper have been eliminated. FIG. 31 shows the new seeding port lid that has the option of filter paper if necessary. FIG. 32 shows how the o-ring fits into the container for the main lid as well as how the ring assemble is attached to the container. FIG. 33 illustrates how the gyroscope rings are attached in order to ensure enough tension to have control over the movements during seeding.

FIGS. 34-35 are images of the stand used to position the bioreactor for seeding. The ball and socket joint is used to angle the bioreactor so the field of vision is optimized when seeding each petal of the scaffold. The black collar rotates to reposition the bioreactor and has a range of 360 degrees.

FIG. 36 shows the most up to date design depicting the change to the lid design. The design has been changed to a clamp-on lid using no threads. The lid is sealed and o-ring compression is achieved using stainless steel rings that are clamped together using 5 knobs.

### DETAILED DESCRIPTION OF THE INVENTION

Methods and constructs useful in the reconstruction, repair, augmentation or replacement of organs or tissues structures are provided. Methods and constructs involved in the establishment of the “all-in-one” closed system bioreactor are also provided.

Bioreactors are used in neo-organ construct production to allow for an appropriate environment for the maintenance of healthy culturing conditions from sterilization to shipment of the neo-organ. The closed system “all-in-one bioreactor” is designed to allow for minimal exposure of the scaffold to the open air in order to maintain sterility. The design allows for the same container to be utilized for sterilization, pre-wetting, cell seeding, medium exchange, and shipment. The “all-in-one” bioreactor also remains completely closed after the urothelial cell seeding step to removal of the neo-organ construct in the surgical theatre for implantation in the patient. This allows for sufficient time for release testing to occur, which is a prerequisite for implantation of the neo-organ construct into the patient.

In its broadest form, the methods and constructs of the present invention are useful in the reconstruction, repair, augmentation or replacement of organs or tissues structures that comprise multilayer cellular organization and particularly those organs or tissue structures that are luminal in nature. More particularly, the present invention provides methods and constructs that facilitate the reconstruction, repair, augmentation or replacement of shaped hollow organs or tissue structures that exhibit a laminar segregation of different cell types and that have a need to retain a general luminal shape. Luminal organs or tissue structures that contain a smooth muscle cell (SMC) layer to impart compliant or contractible properties to the organ or structure are particularly well suited to the methods and constructs of the present invention.

In an example of one preferred embodiment of the invention, the luminal organ is the bladder, which has an inner layer of a first cell population that comprises urothelial cells and an outer layer of a second cell population that comprises smooth muscle cells. This organization is also present in other genitourinary organs and tissue structures such as the ureters and urethra. Laminarily organized organs or tissues refer to any organ or tissue made up of, or arranged in laminae including ductal tissue. Other suitable laminarily organized luminal organs, tissue structure, or ductal tissues to which the present invention is directed include vas deferens, fallopian tubes, lacrimal ducts, trachea, stomach, intestines, vasculature, biliary duct, ductus ejaculatorius, ductus epididymidis, ductus parotideus, and surgically created shunts.

The neo-organ constructs and methods of the present invention comprise a biocompatible synthetic or natural polymeric matrix or scaffold, and one or more cell populations seeded on one or more surfaces of the matrix or scaffold. The method of the present invention in its broadest aspect encompasses as a first step providing a biocompatible synthetic or natural polymeric matrix or scaffold that is shaped to conform to its use as a part or all of the luminal organ or tissue structure to be repaired, reconstructed, augmented or replaced. Hereinafter, the terms matrix and scaffold may be used interchangeably. A biocompatible material is any substance not having toxic or injurious effects on biological function. The shaped matrix or scaffold is preferably porous to allow for cell deposition both on and in the pores of the matrix. The shaped matrix or scaffold may then be contacted with one or more cell populations to seed the cell populations on or into (or both) the matrix or scaffold. The cell-seeded matrix scaffold (i.e., the construct) is then implanted in the body of the recipient where the construct facilitates the regeneration of neo-organs or tissue structures. The constructs may be used to reconstruct, repair, augment or replace any organ, and may especially be utilized in patients having a defect in urogenital tissues such as the bladder.

In a preferred embodiment, the materials and methods of the invention are useful for the reconstruction, replacement or augmentation of bladder tissue. Thus, the invention provides treatments for such conditions as neurogenic bladder, bladder exstrophy, bladder volume insufficiency, bladder non-compliance, reconstruction of bladder following partial or total cystectomy, repair of bladders damaged by trauma, and the like.

While reference is made herein to replacement or augmentation of bladder according to the invention, it will be understood that the methods and materials of the invention are useful for tissue reconstruction, replacement or augmentation of a variety of tissues and organs in a subject. Thus, for example, organs or tissues such as bladder, ureter, urethra, renal pelvis, and the like, can be augmented or repaired with polymeric matrixes seeded with cells. The materials and methods of the invention further can be applied to the reconstruction, replacement or augmentation of vascular tissue (see, e.g., Zdrahala, R. J., J. Biomater. Appl. (4): 309-29 (1996)), intestinal tissues, stomach (see, e.g., Laurencin, C. T. et al., J Biomed Mater. Res. 30 (2): 133-8 1996), and the like. The patient to be treated may be of any species of mammals such as a dog, cat, pig, horse, cow, or human, in need of reconstruction, repair, replacement or augmentation of a tissue.

Neo-Organ Matrices or Scaffolds

Biocompatible material and especially biodegradable material is the preferred material for the construction of the neo-organ matrix or scaffold.

Biocompatible refers to materials which do not have toxic or injurious effects on biological functions. Biodegradable refers to material that can be absorbed or degraded in a patient's body. Representative materials for forming the biodegradable matrix or scaffold include natural or synthetic polymers, such as, for example, collagen, poly(alpha esters) such as poly(lactate acid) and poly(glycolic acid), polyorthoesters and polyanhydrides and their copolymers, which degrade by hydrolysis at a controlled rate and are reabsorbed. These materials provide the maximum control of degradability, manageability, size and configuration. Preferred biodegradable polymer material includes polyglycolic acid and polyglactin, developed as absorbable synthetic material. Polyglycolic acid and polyglactin fibers may be used as supplied by the manufacturer. Other biodegradable materials include cellulose ether, cellulose, cellulosic ester, fluorinated polyethylene, phenolic, poly-4-methylpentene, polyacrylonitrile, polyamide, polyamideimide, polyacrylate, polybenzoxazole, polycarbonate, polycyanoarylether, polyester, polyestercarbonate, polyether, polyetheretherketone, polyetherimide, polyetherketone, polyethersulfone, polyethylene, polyfluoroolefin, polyimide, polyolefin, polyoxadiazole, polyphenylene oxide, polyphenylene sulfide, polypropylene, polystyrene, polycaprolactone, polysulfide, polysulfone, polytetrafluoroethylene, polythioether, polytriazole, polyurethane, polyvinyl, polyvinylidene fluoride, regenerated cellulose, silicone, urea-formaldehyde, or copolymers or physical blends of these materials. The material may be impregnated with suitable antimicrobial agents and may be colored by a color additive to improve visibility and to aid in surgical procedures.

Other biocompatible materials include synthetic suture material manufactured by Ethicon Co. (Ethicon Co., Somerville, N.J.), such as MONOCRYL® (copolymer of glycolide and epsilon-caprolactone), VICRYL® or Polyglactin 910 (copolymer of lactide and glycolide coated with Polyglactin 370 and calcium stearate), and PANACRYL® (copolymer of lactide and glycolide coated with a polymer of caprolactone and glycolide). (Craig P. H., Williams J. A., Davis K. W., et al.: A Biological Comparison of Polyglactin 910 and Polyglycolic Acid Synthetic Absorbable Sutures. Surg. 141; 1010, (1975)) and polyglycolic acid. These materials can be used as supplied by the manufacturer.

In yet another embodiment, the matrix or scaffold can be created using parts of a natural decellularized organ. Biostructures, or parts of organs can be decellularized by removing the entire cellular and tissue content from the organ. The decellularization process comprises a series of sequential extractions. One key feature of this extraction process is that harsh extraction that may disturb or destroy the complex infra-structure of the biostructure, be avoided. The first step involves removal of cellular debris and solubilization of the cell membrane. This is followed by solubilization of the nuclear cytoplasmic components and the nuclear components.

Preferably, the biostructure, e.g., part of an organ is decellularized by removing the cell membrane and cellular debris surrounding the part of the organ using gentle mechanical disruption methods. The gentle mechanical disruption methods must be sufficient to disrupt the cellular membrane. However, the process of decellularization should avoid damage or disturbance of the biostructure's complex infra-structure. Gentle mechanical disruption methods include scraping the surface of the organ part, agitating the organ part, or stirring the organ in a suitable volume of fluid, e.g., distilled water. In one preferred embodiment, the gentle mechanical disruption method includes stirring the organ part in a suitable volume of distilled water until the cell membrane is disrupted and the cellular debris has been removed from the organ.

After the cell membrane has been removed, the nuclear and cytoplasmic components of the biostructure are removed. This can be performed by solubilizing the cellular and nuclear components without disrupting the infra-structure. To solubilize the nuclear components, non-ionic detergents or surfactants may be used. Examples of nonionic detergents or surfactants include, but are not limited to, the Triton series, available from Rohm and Haas of Philadelphia, Pa., which includes Triton X-100, Triton N-101, Triton X-114, Triton X-405, Triton X-705, and Triton DF-16, available commercially from many vendors; the Tween series, such as monolaurate (Tween 20), monopalmitate (Tween 40), monooleate (Tween 80), and polyoxethylene-23-lauryl ether (Brij. 35), polyoxyethylene ether W-1 (Polyox), and the like, sodium cholate, deoxycholates, CHAPS, saponin, n-Decyl-D-glucopuranoside, n-heptyl-D-glucopyranoside, n-Octyl-D-glucopyranoside and Nonidet P-40.

One skilled in the art will appreciate that a description of compounds belonging to the foregoing classifications, and vendors may be commercially obtained and may be found in “Chemical Classification, Emulsifiers and Detergents”, McCutcheon's, Emulsifiers and Detergents, 1986, North American and International Editions, McCutcheon Division, MC Publishing Co., Glen Rock, N.J., U.S.A. and Judith Neugebauer, A Guide to the Properties and Uses of Detergents in Biology and Biochemistry, Calbiochem. R., Hoechst Celanese Corp., 1987. In one preferred embodiment, the non-ionic surfactant is the Triton. series, preferably, Triton X-100.

The concentration of the non-ionic detergent may be altered depending on the type of biostructure being decellularized. For example, for delicate tissues, e.g., blood vessels, the concentration of the detergent should be decreased. Preferred concentration ranges of non-ionic detergent can be from about 0.001 to about 2.0% (w/v). More preferably, about 0.05 to about 1.0% (w/v). Even more preferably, about, 0.1% (w/v) to about 0.8% (w/v). Preferred concentrations of these range from about 0.001 to about 0.2% (w/v), with about 0.05 to about 0.1% (w/v) particular preferred.

The cytoskeletal component, which includes the dense cytoplasmic filament networks, intercellular complexes and apical microcellular structures, may be solubilized using alkaline solution, such as, ammonium hydroxide. Other alkaline solution consisting of ammonium salts or their derivatives may also be used to solubilize the cytoskeletal components. Examples of other suitable ammonium solutions include ammonium sulphate, ammonium acetate and ammonium hydroxide. In a preferred embodiment, ammonium hydroxide is used.

The concentration of the alkaline solutions, e.g., ammonium hydroxide, may be altered depending on the type of biostructure being decellularized. For example, for delicate tissues, e.g., blood vessels, the concentration of the detergent should be decreased. Preferred concentrations ranges can be from about 0.001 to about 2.0% (w/v). More preferably, about 0.005 to about 0.1% (w/v). Even more preferably, about, 0.01% (w/v) to about 0.08% (w/v).

The decellularized, lyophilized structure may be stored at a suitable temperature until required for use. Prior to use, the decellularized structure can be equilibrated in suitable isotonic buffer or cell culture medium. Suitable buffers include, but are not limited to, phosphate buffered saline (PBS), saline, MOPS, HEPES, Hank's Balanced Salt Solution, and the like. Suitable cell culture medium includes, but is not limited to, RPMI 1640, Fisher's, Iscove's, McCoy's, Dulbecco's medium, and the like.

Still other biocompatible materials that may be used include stainless steel, titanium, silicone, gold and silastic.

The biocompatible polymer may be shaped using methods such as, for example, solvent casting, compression molding, filament drawing, meshing, leaching, weaving and coating. In solvent casting, a solution of one or more polymers in an appropriate solvent, such as methylene chloride, is cast as a branching pattern relief structure. After solvent evaporation, a thin film is obtained. In compression molding, a polymer is pressed at pressures up to 30,000 pounds per square inch into an appropriate pattern. Filament drawing involves drawing from the molten polymer and meshing involves forming a mesh by compressing fibers into a felt-like material. In leaching, a solution containing two materials is spread into a shape close to the final form of the construct. Next a solvent is used to dissolve away one of the components, resulting in pore formation. (See Mikos, U.S. Pat. No. 5,514,378, hereby incorporated by reference.) In nucleation, thin films in the shape of a RUG are exposed to radioactive fission products that create tracks of radiation damaged material. Next the polycarbonate sheets are etched with acid or base, turning the tracks of radiation-damaged material into pores. Finally, a laser may be used to shape and burn individual holes through many materials to form a structure with uniform pore sizes. Coating refers to coating or permeating a polymeric structure with a material such as, for example liquefied copolymers (poly-DL-lactide co-glycolide 50:50 80 mg/ml methylene chloride) to alter its mechanical properties. Coating may be performed in one layer, or multiple layers until the desired mechanical properties are achieved. These shaping techniques may be employed in combination, for example, a polymeric matrix or scaffold may be weaved, compression molded and glued together. Furthermore different polymeric materials shaped by different processes may be joined together to form a composite shape. The composite shape may be a laminar structure. For example, a polymeric matrix or scaffold may be attached to one or more polymeric matrixes to form a multilayer polymeric matrix or scaffold structure. The attachment may be performed by gluing with a liquid polymer or by suturing. In addition, the polymeric matrix or scaffold may be formed as a solid block and shaped by laser or other standard machining techniques to its desired final form. Laser shaping refers to the process of removing materials using a laser.

The polymeric matrix or scaffold can be reinforced. For example, reinforcing materials may be added during the formation of a synthetic matrix or scaffold or attached to the natural or synthetic matrix prior to implantation. Representative materials for forming the reinforcement include natural or synthetic polymers, such as, for example, collagen, poly(alpha esters) such as poly(lactate acid), poly(glycolic acid), polyorthoesters and polyanhydrides and their copolymers, which degraded by hydrolysis at a controlled rate and are reabsorbed. These materials provide the maximum control of degradability, manageability, size and configuration.

The biodegradable polymers can be characterized with respect to mechanical properties, such as tensile strength using an Instron tester, for polymer molecular weight by gel permeation chromatography (GPC), glass, transition temperature by differential scanning calorimetry (DSC) and bond structure by infrared (IR) spectroscopy; with respect to toxicology by initial screening tests involving Ames assays and in vitro teratogenicity assays and implantation studies in animals for immunogenicity, inflammation, release and degradation studies. In vitro cell attachment and viability can be assessed using scanning electron microscopy, histology and quantitative assessment with radioisotopes. The biodegradable material may also be characterized with respect to the amount of time necessary for the material to degrade when implanted in a patient. By varying the construction, such as, for example, the thickness and mesh size, the biodegradable material may substantially biodegrade between about 2 years or about 2 months, preferably between about 18 months and about 4 months, most preferably between about 15 months and about 8 months and most preferably between about 12 months and about 10 months. If necessary, the biodegradable material may be constructed so as not to degrade substantially within about 3 years, or about 4 years or about five or more years.

The polymeric matrix or scaffold may be fabricated with controlled pore structure as described above. The size of the pores may be used to determine the cell distribution. For example, the pores on the polymeric matrix or scaffold may be large to enable cells to migrate from one surface to the opposite surface. Alternatively, the pores may be small such that there is fluid communication between the two sides of the polymeric matrix or scaffold but cells cannot pass through. Suitable pore size to accomplish this objective may be about 0.04 micron to about 10 microns in diameter, preferably between about 0.4 micron to about 4 microns in diameter. In some embodiments, the surface of the polymeric matrix or scaffold may comprise pores sufficiently large to allow attachment and migration of a first population of cells into the pores. The pore size may be reduced in the interior of the polymeric matrix or scaffold to prevent cells from migrating from one side of the polymeric matrix or scaffold to the opposite side. On the opposite side of the polymeric matrix, the pores may again enlarge to allow the attachment and establishment of a second population of cells. Because of the reduced pore size in the interior of the polymeric matrix, the first cell population and the second cell population initially cannot mix. One embodiment of a polymeric matrix or scaffold with reduced pore size is a laminated structure of a small pore material sandwiched between two large pore material. Alternatively, a large pore material laminated to a small pore material may also allow cells to establish growth on both sides without any intermixing of cells. Polycarbonate membranes are especially suitable because they can be fabricated in very controlled pore sizes such as, for example, about 0.01 microns, about 0.05 micron, about 0.1 micron, about 0.2 micron, about 0.45 micron, about 0.6 micron, about 1.0 micron, about 2.0 microns and about 4.0 microns. At the submicron level the polymeric matrix or scaffold may be impermeable to bacteria, viruses and other microbes.

Optimally, the matrix or scaffold should be shaped such that after its biodegradation, the resulting reconstructed bladder is collapsible when empty in a fashion similar to a natural bladder and the ureters will not be obstructed while the urinary catheter has been removed from the tissue engineered bladder without leaving a leak point from the dome. The bioengineered bladder construct can be produced as one piece or each part can be individually produced or combinations of the sections can be produced as specific parts. Each specific matrix or scaffold part may be produced to have a specific function. Otherwise specific parts may be produced for manufacturing ease. Specific parts may be constructed of specific materials and may be designed to deliver specific properties. Specific part properties may include tensile strength similar to the native tissue (e.g. ureters) of 0.5 to 1.5 MPa² and an ultimate elongation of 30 to 100% or the tensile strength may range from 0.5 to 28 MPa², ultimate elongations may range from 10-200% and compression strength may be 12.

A mesh-like structure formed of fibers, which may be round, scalloped, flattened, star shaped, solitary or entwined with other fibers is preferred. The use of branching fibers is based upon the same principles which nature has used to solve the problem of increasing surface area proportionate to volume increases. All multicellular organisms utilize this repeating branching structure. Branching systems represent communication networks between organs, as well as the functional units of individual organs. Seeding and implanting this configuration with cells allows implantation of large numbers of cells, each of which is exposed to the environment of the host, providing for free exchange of nutrients and waste while neovascularization is achieved. The polymeric matrix or scaffold may be made flexible or rigid, depending on the desired final form, structure and function.

Polymeric matrixes can be treated with additives or drugs prior to implantation (before or after the polymeric matrix or scaffold is seeded with cells, if the optional seeded cells are employed), e.g., to promote the regeneration of new tissue after implantation. Thus, for example, growth factors, cytokines, extracellular matrix components, and other bioactive materials can be added to the polymeric matrix or scaffold to promote graft healing and formation of new tissue. Such additives will in general be selected according to the tissue or organ being reconstructed, repaired or augmented, to ensure that appropriate new tissue is regenerated in the engrafted organ or tissue (for examples of such additives for use in promoting bone healing, see, e.g., Kirker-Head, C. A. Vet. Surg. 24 (5): 408-19 (1995)). For example, when polymeric matrices (optionally seeded with endothelial cells) are used to augment vascular tissue, vascular endothelial growth factor (VEGF), (see, e.g., U.S. Pat. No. 5,654,273) can be employed to promote the regeneration of new vascular tissue. Growth factors and other additives (e.g., epidermal growth factor (EGF), heparin-binding epidermal-like growth factor (HBGF), fibroblast growth factor (FGF), cytokines, genes, proteins, and the like) can be added in amounts in excess of any amount of such growth factors (if any) which may be produced by the cells seeded on the polymeric matrix or scaffold, if added cells are employed. Such additives are preferably provided in an amount sufficient to promote the regeneration of new tissue of a type appropriate to the tissue or organ, which is to be repaired, replaced or augmented (e.g., by causing or accelerating infiltration of host cells into the graft). Other useful additives include antibacterial agents such as antibiotics.

One preferred supporting matrix or scaffold is composed of crossing filaments which can allow cell survival by diffusion of nutrients across short distances once the cell support matrix or scaffold is implanted. The cell support matrix or scaffold becomes vascularized in concert with expansion of the cell mass following implantation.

The building of three-dimensional structure constructs in vitro, prior to implantation, facilitates the eventual terminal differentiation of the cells after implantation in vivo, and minimizes the risk of an inflammatory response towards the matrix or scaffold, thus avoiding graft contracture and shrinkage.

The polymeric matrix or scaffold may be sterilized using any known method before use. The method used depends on the material used in the polymeric matrix or scaffold. Examples of sterilization methods include steam, dry heat, radiation, gases such as ethylene oxide, gas and boiling.

Method for Forming Neo-Organ Matrices or Scaffolds

The biocompatible scaffold may be shaped using methods such as, for example, solvent casting, compression molding, filament drawing, meshing, leaching, weaving, foaming, electrospinning and coating. In solvent casting, a solution of one or more polymers in an appropriate solvent, such as methylene chloride, is cast as a branching pattern relief structure. After solvent evaporation, a thin film is obtained. In compression molding, a polymer is pressed at pressures up to 30,000 pounds per square inch into an appropriate pattern. Filament drawing involves drawing from the molten polymer and meshing involves forming a mesh by compressing fibers into a felt-like material. In leaching, a solution containing two materials is spread into a shape close to the final form of the artificial organ. Next a solvent is used to dissolve away one of the components, resulting in pore formation. (See U.S. Pat. No. 5,514,378 to Mikos).

In nucleation, thin films in the shape of an artificial organ are exposed to radioactive fission products that create tracks of radiation damaged material. Next the polycarbonate sheets are etched with acid or base, turning the tracks of radiation-damaged material into pores. Finally, a laser may be used to shape and burn individual holes through many materials to form a scaffold structure with uniform pore sizes. Coating refers to coating or permeating a structure with a material such as, for example liquefied copolymers (poly-DL-lactide co-glycolide 50:50 80 mg/ml methylene chloride) to alter its mechanical properties. Coating may be performed in one layer, or multiple layers until the desired mechanical properties are achieved. These shaping techniques may be employed in combination, for example, a scaffold may be weaved, compression molded and glued together. Furthermore different materials shaped by different processes may be joined together to form a composite shape. The composite shape may be a laminar structure. For example, a matrix or scaffold may be attached to one or more matrices to form a multilayer scaffold structure. The attachment may be performed by gluing with a liquid polymer or by suturing. In addition, the matrix or scaffold may be formed as a solid block and shaped by laser or other standard machining techniques to its desired final form. Laser shaping refers to the process of removing materials using a laser.

The scaffold may be shaped into any number of desirable configurations to satisfy any number of overall system, geometry or space restrictions. For example, in the use of the scaffold for bladder, urethra, valve, or blood vessel reconstruction, the matrix or scaffold may be shaped to conform to the dimensions and shapes of the whole or a part of the tissue.

Naturally, the scaffold may be shaped in different sizes and shapes to conform to the organs of differently sized patients. For bladders, the scaffold should be shaped such that after its biodegradation, the resulting reconstructed bladder may be collapsible when empty in a fashion similar to a natural bladder. The matrix or scaffold may also be shaped in other fashions to accommodate the special needs of the patient.

Cells for Organ Reconstruction

In one embodiment, the scaffolds are seeded with one or more populations of cells to form an artificial organ construct. The artificial organ construct can be autologous, where the cell populations are derived from the subject's own tissue, or allogenic, where the cell populations are derived from another subject within the same species as the patient. The artificial organ construct can also be xenogenic, where the different cell populations are derived form a mammalian species that is different from the subject. For example the cells can be derived from organs of mammals such as humans, monkeys, dogs, cats, mice, rats, cows, horses, pigs, goats and sheep.

The process for isolating cells is described generally, and specific procedures are presented in the Examples provided below. Cells can be isolated from a number of sources, for example, from biopsies, or autopsies. The isolated cells are preferably autologous cells, obtained by biopsy from the subject. For example, a biopsy of skeletal muscle from the arm, forearm, or lower extremities, or smooth muscle from the area treated with local anesthetic with a small amount of lidocaine injected subcutaneously, and expanded in culture. The biopsy can be obtained using a biopsy needle, a rapid action needle which makes the procedure quick and simple. The small biopsy core of either skeletal or smooth muscle can then be expanded and cultured, as described by Atala, et al., (1992) J. Urol. 148, 658-62; Atala, et al. (1993) J. Urol. 150: 608-12. Cells from relatives or other donors of the same species can also be used with appropriate immunosuppression.

Methods for the isolation and culture of cells are discussed in Fauza et al. (1998) J. Ped. Surg. 33, 7-12, incorporated herein by reference. Cells may be isolated using techniques known to those skilled in the art. For example, the tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase and dispase. Mechanical disruption can also be accomplished by a number of methods including, but not limited to, scraping the surface of the organ, the use of grinders, blenders, sieves, homogenizers, pressure cells, or insonicators. For a review of tissue disaggregation techniques, see Freshney, (1987), Culture of Animal Cells. A Manual of Basic Technique, 2d Ed., A. R. Liss, Inc., New York, Ch. 9, pp. 107-126.

Preferred cell types include, but are not limited to, urothelial cells, mesenchymal cells, especially smooth or skeletal muscle cells, myocytes (muscle stem cells), fibroblasts, chondrocytes, adipocytes, fibromyoblasts, and ectodermal cells, including ductile and skin cells, hepotocytes, Islet cells, cells present in the intestine, and other parenchymal cells, osteoblasts and other cells forming bone or cartilage. In some cases, it may also be desirable to include nerve cells.

Once the tissue has been reduced to a suspension of individual cells, the suspension can be fractionated into subpopulations from which the cells elements can be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and magnetic-activated and fluorescence-activated cell sorting. For a review of clonal selection and cell separation techniques, see Freshney, (1987), Culture of Animal Cells. A Manual of Basic Techniques, 2d Ed., A. R. Liss, Inc., New York, Ch. 11 and 12, pp. 137-168. For example, one cell type may be enriched by fluorescence-activated cell sorting, and other cell types may be reduced for collection of a specific cell type.

Cell fractionation may also be desirable, for example, when the donor has diseases such as cancer or metastasis of other tumors to the desired tissue. A cell population may be sorted to separate malignant cells or other tumor cells from normal noncancerous cells. The normal noncancerous cells, isolated from one or more sorting techniques, may then be used for organ reconstruction.

Isolated cells can be cultured in vitro to increase the number of cells available for coating the biocompatible scaffold. The use of allogenic cells, and more preferably autologous cells, is preferred to prevent tissue rejection. However, if an immunological response does occur in the subject after implantation of the artificial organ, the subject may be treated with immunosuppressive agents such as, cyclosporin or FK506, to reduce the likelihood of rejection. In certain embodiments, chimeric cells, or cells from a transgenic animal, can be coated onto the biocompatible scaffold.

Isolated cells may be transfected prior to coating with genetic material. Useful genetic material may be, for example, genetic sequences which are capable of reducing or eliminating an immune response in the host. For example, the expression of cell surface antigens such as class I and class II histocompatibility antigens may be suppressed. This may allow the transplanted cells to have reduced chance of rejection by the host. In addition, transfection could also be used for gene delivery.

Isolated cells can be normal or genetically engineered to provide additional or normal function. Methods for genetically engineering cells with retroviral vectors, polyethylene glycol, or other methods known to those skilled in the art can be used. These include using expression vectors which transport and express nucleic acid molecules in the cells. (See Goeddel; Gene Expression Technology: Methods in Enzymology 185, Academic Press, San Diego, Calif. (1990).

Vector DNA is introduced into prokaryotic or cells via conventional transformation or transfection techniques. Suitable methods for transforming or transfecting host cells can be found in Sambrook et al. (Molecular Cloning: A Laboratory Manual, 2nd Edition, Cold Spring Harbor Laboratory press (1989)), and other laboratory textbooks.

Seeding of the Neo-Organ Matrix or Scaffold

Seeding of cells onto the matrix or scaffold can be performed according to standard methods. For example, the seeding of cells onto polymeric substrates for use in tissue repair has been reported (see, e.g., Atala, A. et al., J. Urol. 148 (2 Pt 2): 658-62 (1992); Atala, A., et al. J. Urol. 150 (2 Pt 2): 608-12 (1993)). Cells grown in culture can be trypsinized to separate the cells, and the separated cells can be seeded on the matrix or scaffold. Alternatively, cells obtained from cell culture can be lifted from a culture plate as a cell layer, and the cell layer can be directly seeded onto the scaffold without prior separation of the cells.

In a preferred embodiment, in the range of 1 million to 700 million cells are suspended in medium and applied to each square centimeter of a surface of a scaffold. Preferably, between 1 million and 50 million cells, and more preferably, between 1 million and 10 million cells are suspended in media and applied to each square centimeter of a surface of a scaffold. The matrix or scaffold is incubated under standard culturing conditions, such as, for example, 37° C., 5% CO₂, for a period of time until the cells attached. Other seeding techniques may also be used depending on the matrix or scaffold and the cells. For example, the cells may be applied to the matrix or scaffold by vacuum filtration. Selection of cell types, and seeding of cells onto a scaffold, will be routine to one of ordinary skill in the art in light of the teachings herein.

In one embodiment, the matrix or scaffold is seed with one population of cells to form an artificial organ construct. In another embodiment, the matrix or scaffold is seeded on two sides with two different populations of cells. This may be performed by first seeding one side of the matrix or scaffold and then seeding the other side. For example, the scaffold may be placed with one side on top and seeded. Then the matrix or scaffold may be repositioned so that a second side is on top. The second side may then be seeded with a second population of cells. Alternatively, both sides of the matrix or scaffold may be seeded at the same time. For example, two cell chambers may be positioned on both sides (i.e., a sandwich) of the scaffold. The two chambers may be filled with different cell populations to seed both sides of the matrix or scaffold simultaneously. The sandwiched scaffold may be rotated, or flipped frequently to allow equal attachment opportunity for both cell populations. Simultaneous seeding may be preferred when the pores of the matrix or scaffold are sufficiently large for cell passage from one side to the other side. Seeding the scaffold on both sides simultaneously will reduce the likelihood that the cells would migrate to the opposite side.

In another embodiment, two separate scaffolds may be seeded with different cell populations. After seeding, the two matrices may be attached together to form a single matrix or scaffold with two different cell populations on the two sides. Attachment of the scaffolds to each other may be performed using standard procedures such as fibrin glue, liquid co-polymers, sutures and the like.

Surgical Reconstruction

Grafting of scaffolds to an organ or tissue to be augmented can be performed according to the methods described in the Examples or according to art-recognized methods. The matrix or scaffold can be grafted to an organ or tissue of the subject by suturing the graft material to the target organ. Implanting a neo-organ construct for total organ replacement can be performed according to art-recognized surgical methods.

The described techniques may also be used to treat cancer in an organ or tissue. For example, a normal tissue sample may be excised from a patient suffering from cancer. Cell populations from the tissue sample may be cultured for a period of time in vitro and expanded. The cells may be sorted using a florescent activated cell sorter to remove cancerous or precancerous cells. The sorted cells may be used to construct a seeded scaffold. At the same time, the patient may be treated for cancer. Cancer treatment may involve excision of the cancerous part of the organ in addition to chemotherapy or radiation treatment. After the cancer treatment, the seeded scaffold may be used to reconstruct the tissue or organ.

While a method for bladder reconstruction is disclosed in the Examples, other methods for attaching a graft to an organ or tissue of the subject (e.g., by use of surgical staples) may also be employed. Such surgical procedures can be performed by one of ordinary skill in the art according to known procedures.

The present invention will be further understood by reference to the following non-limiting examples.

### EXAMPLE 1

### Creation of Bladder-Shaped Polymers

The neo-organ constructs described herein are presented using neo-bladder constructs as an example. While reference is made here to neo-bladder constructs, it will be understood that the methods and materials described herein are useful for creating a variety of neo-organs and neo-vessel augmentation constructs, including, for example, neo-kidney augmentation constructs.

Manufacture of the neobladder matrix or scaffold. The neobladder matrices or scaffolds are constructed using polyglycolide-polyglycolic acid (PGA) non-woven felt (BMS or Concordia 2.5 mm thick, 58 mg/cc or 99 mg/ml). The PGA non-woven felt is cut using a neo-bladder pattern as a template. The neo-bladder pattern is for example, spherical, quasi-spherical, hemispherical, or quasi-hemispherical in shape, such that bladder repair, or augmentation procedures require one hemispherical or quasi-hemispherical neo-bladder construct, while total bladder reconstruction may require one spherical or quasi-spherical neo-bladder construct, or two hemispherical or quasi-hemispherical neo-bladder constructs joined together to create a spherical or quasi-spherical construct.

To create spherical, quasi-spherical, hemispherical or quasi-hemispherical neo-bladder constructs for repair, augmentation, or replacement, the PGA non-woven felt is cut using a neo-bladder template. The neo-bladder template is a single piece of PGA non-woven felt or multiple pieces that are joined together, e.g., two or more pieces, three or more pieces, or four or more pieces. The template is then assembled, for example, by joining distinct areas of a single template together, or by joining two or more pieces of a multi-piece template together.

In one embodiment, a single distinct template is used to form a spherical or quasi-spherical neo-bladder construct. In another embodiment, a single distinct template is used to form two hemispherical or quasi-hemispherical neo-bladder constructs, such that a two-part construct is initially formed from one integral part. In another embodiment, two or more distinct templates are used to create hemispherical or quasi-hemispherical neo-bladder constructs which are adapted to mate to each other, such that each half of the neo-bladder construct is formed from two or more distinct parts. In some embodiments, the two or more distinct templates or parts used to create a hemispherical or quasi-hemispherical parts adapted to mate are symmetrical, while in other embodiments, the two or more distinct templates or parts are asymmetrical.

Augmentation Construct Designs

Single neo-bladder template designs, when assembled, produce a spherical or quasi-spherical construct for use in bladder augmentation. Regardless of the template used, the assembled construct is designed to fit within the geometry of the intended site of implantation, e.g., within a human subject.

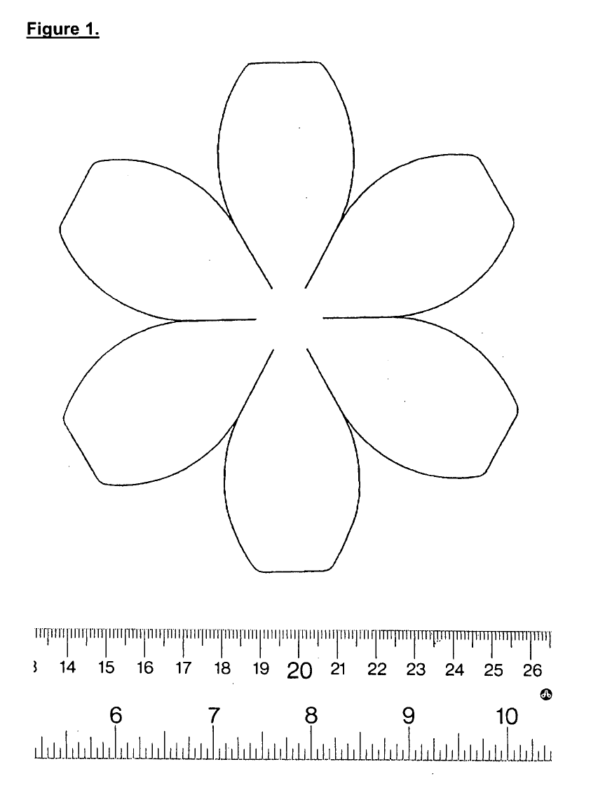

An example of an initial, single neo-bladder template used to create a quasi-spherical neo-bladder construct is shown in FIG. 1. The neo-bladder template of FIG. 1, when assembled, creates a unitary construct that is spherical or quasi-spherical. After the PGA non-woven felt is die-cut or manually pressed using the pattern shown in FIG. 1, the petal portions are mated together. The petal portions can be mated using glue, staples, sutures or other technique known to one of ordinary skill in the art. For example, a 4-0 vicryl suture is used to suture each petal together from the inside out, using a simple uninterrupted stitch or “blanket stitch” with a knot every third or fourth stitch. Once two petals are sutured together, loops of suture, e.g., a 1.5 inch loop or a 3 inch loop, are made at the end of every other petal. Preferably, there are six loops per scaffold, one at the end of each petal. Another loop of suture, e.g., a three inch loop, is made at the apex of the scaffold to finish the suturing. These loops form handles for increased ease of manipulation and implantation for the neobladder constructs described herein. For example, the surgeon uses these loops as handles to hold onto the neobladder construct during implantation.

In other embodiments, the neobladder matrix or scaffold is formed using any of a variety of techniques known in the art. The neobladder matrix or scaffold is, for example, molded, foamed or electrospun.

Neo-organ matrix or scaffold prewetting, coating, and sterilization. The neo-organ scaffold is prewetted, coated and sterilized using techniques readily ascertainable to those skilled in the art.

Pre-wetting of neobladder scaffold prior to cell harvesting. Prior to cell harvesting, e.g., one day prior to harvesting cells, a sterilized scaffold undergoes a pre-wetting procedure. The scaffold is pre-wet by adding 500 ml of SMC growth medium (described below) to a pre-wetting container in which the scaffold is placed, such as a sterilized 1 liter NALGENE® polypropylene jar with a screw cap lid with a Teflon seal.

### EXAMPLE 2

### Cell Harvest and Culture

Biopsy procurement. In contrast to previous studies in which a 1×1 cm biopsy was taken from the side of the bladder using a scalpel to dissociate the tissue, the tissue samples used to create the neobladder constructs described in this Example were obtained by taking a 1×1 cm biopsy from the bladder apex, using a staple method. Previous biopsy procedures, such as the methods described in U.S. Pat. No. 6,576,019 by Atala et al., removed tissue from the vesical dome in general. In contrast, the biopsy procedures used herein remove tissue from a specific portion of the vesical dome, the bladder apex. Removing tissue from the bladder apex has been shown to provide a greater yield of useful cells. Useful cells refers to viable cells that are capable of expansion and seeding on the neobladder scaffolds described herein.

The staple method used herein involves making a loop in the apex of the bladder, stapling the base of the loop, and excising the loop. The staple biopsy provides several advantages over a scalpel biopsy, including, for example, an increase in the amount of tissue safely removed and a concomitant decrease in deleterious effects for the subject. Cells isolated from biopsy material procured in this manner demonstrated superior in vitro attachment and proliferation compared to cells isolated from biopsies obtained from the bladder side using a scalpel. The biopsy material is transported in a standard culture medium such as DMEM supplemented with antibiotic to decrease the incidence of receiving contaminated biopsy specimens. All subsequent manipulations on the biopsy sample are performed under aseptic conditions, e.g., within the confines of a biosafety cabinet (BSC). Urothelial and smooth muscle cell populations, dissociated from the bladder biopsies, are routinely expanded and passaged separately.

### EXAMPLE 3

### Cell Seeding on Polymeric Matrix or Scaffold

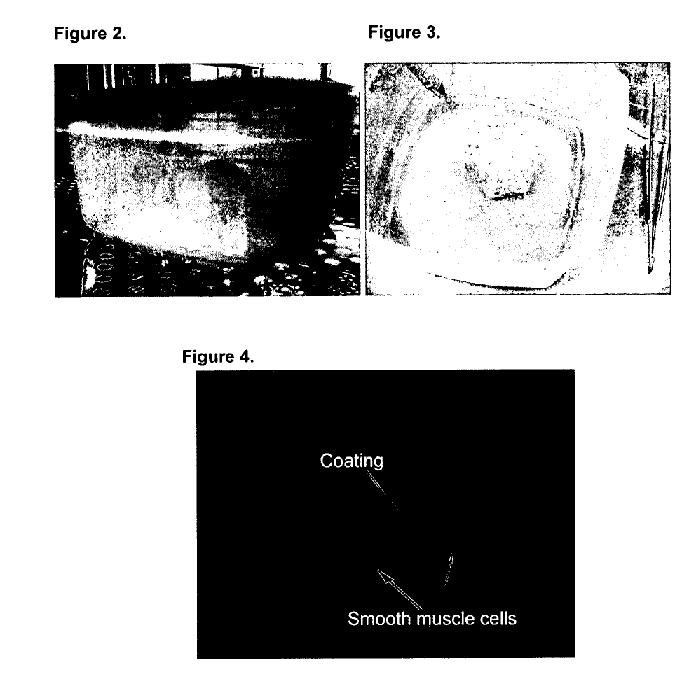

Neo-bladder matrix or scaffold seeding with SMC. After the smooth muscle cells (SMC) are harvested and expanded as described above in Example 2, the cell pellet is resuspended in 6 ml of SMC growth medium. The matrix or scaffold is removed from the pre-wetting container using forceps and is placed in an empty sterile cell-seeding container (see FIGS. 2 and 3, originally designed and manufactured by Tengion Inc.). The cells are distributed evenly on the outside surface of the scaffold.

Bright field microscopy (FIG. 4) confirmed that SMC do indeed take up residence within scaffolds seeded using the procedures described above.

Neobladder scaffold seeding with Urothelial Cells. After the urothelial cells (UC) are harvested and expanded, the cell pellet is resuspended in 6 ml of Construct Growth Medium 1:1 mixture of DMEM/10% FBS:KSFM). The cells are distributed evenly on the inside surface of the scaffold (FIG. 5).

### EXAMPLE 4

### Packaging and Shipping of Cell Seeded Neobladder Constructs

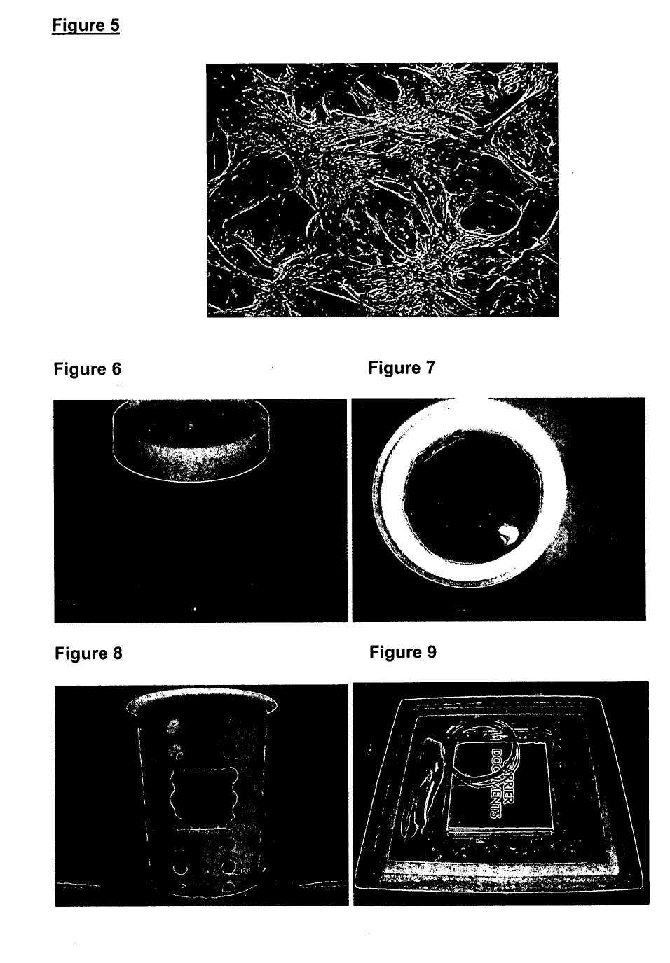

Once the cell-seeded neobladder construct has incubated in the bioreactor for 6 days, it is transported to the shipping container. Initially, the shipping container was a 1 liter NALGENE® polypropylene jar with a screw cap lid with a Teflon seal (FIG. 6). The NALGENE® jar contained an inner plastic basket which supported the neo-organ during transport (FIG. 7). The neo-organ could be secured to the inner support basket to prohibit movement during the shipping process (FIG. 8). The inner basket could be removed at time of surgery. This enabled the surgical team to remove the neo-organ from the outside container, drain the medium, and then place the sterile neo-organ basket onto the surgical field. This shipping container was an original design and was manufactured by Tengion, Inc. The NALGENE® shipping container was chosen for its size and volume requirements necessary for shipping. During shipping, the container was sealed and two layers of parafilm are wrapped around the edge of the lid to prohibit leakage. The shipping container was labeled and placed in a temperature controlled insulated box, sealed, and shipped (FIG. 9).

### EXAMPLE 5

### Bladder Reconstruction

Following pretreatment with intramuscular injection of 0.1 mg of acepromazine for every kilogram of body weight, surgery is performed under inhalational anesthesia (fluorothane) of about 25 to about 35 mg per kilogram of body weight with endotracheal aeration. About 500 mg of Cefazolin sodium is administered intravenously both preoperatively and intraoperatively. Additional treatment of subcutaneously Cefazolin sodium is administered for 5 postoperative days at a dose of about 30 milligrams per kilogram body weight per day. Postoperative analgesic treatment is managed with subcutaneous injections of about 0.1 to about 0.6 milligrams of butorphanol per kilogram of body weight.

A midline laparotomy is performed, the bladder is exposed and both ureters are identified. The bladder wall is incised ventrally and both ureteric junctions are visualized and temporarily intubated with 4 F stents. A subtotal cystectomy is performed, sparing the trigone area bearing the urethra and ureteral junctions. The animals can receive either a bladder shaped polymer alone or a bladder shaped polymer coated with cells. A 10 F silicone catheter is inserted into the urethra from the trigone in a retrograde fashion. An 8 F suprapubic catheter is brought into the bladder lumen passing through a short submucosal tunnel in the trigonal region. The suprapubic catheter is secured to the bladder serosa with a pursestring suture of 4-0 chromic. The anastomosis between trigone and graft is marked at each quadrant with permanent polypropylene sutures for future graft site identification. To ensure adherence between the cell-seeded neobladder construct and the surrounding omentum tissue at the site of implantation and to ensure adherence within the omentum itself, fibrin glue is applied to the surrounding omentum. Alternatively, or in addition, the neo-bladder is covered with fibrin glue (Vitex Technologies Inc., New York, N.Y.). The omentum is wrapped and secured around the neo-reservoir. The abdomen is closed with three layers of 3-0 vicryl. After recovery from anesthesia, all animals wear restraint collars to avoid wound and catheter manipulation during the early postoperative period. The transurethral catheters are removed between postoperative days 4 and 7. Cystograms are performed about four weeks postoperatively, immediately prior to the suprapubic catheter removal. Cystograms and urodynamic studies are serially performed at about 1, about 2, about 3, about 4, about 6 and about 11 months after surgery.

### EXAMPLE 6

### Analysis of Reconstructed Bladder

Urodynamic studies and radiographic cystograms are performed preoperatively and postoperatively at about 1, about 2, about 3, about 4, about 6, and about 11 months after surgery. Animals are sacrificed at about 1, about 2, about 3, about 4, about 6 and about 11 months after surgery. Bladders are retrieved for gross, histological and immunocytochemical analyses.

Urodynamic studies are performed using a 7 F double-lumen transurethral catheter.

The bladders are emptied and intravesical pressures are recorded during instillation of prewarmed saline solution at constant rates. Recordings are continued until leak point pressures (LPP) were reached. Bladder volume at capacity (Volₘₐₓ), LPP and bladder compliance (Volₘₐₓ/LPP) are documented. Bladder compliance, also called bladder elastance, denotes the quality of yielding to pressure or force without disruption. Bladder compliance is also an expression of the measure of the ability to yield to pressure or force without disruption, as an expression of the distensibility of the bladder. It is usually measured in units of volume change per unit of pressure change. Subsequently, radiographic cystograms are performed. The bladders are emptied and contrast medium is instilled intravesically under fluoroscopic control.

### EXAMPLE 7

### Gross Findings

At the intended time points, the animals are euthanized by intravenous pentobarbital administration The internal organs and the urogenital tract are inspected for gross abnormalities. The bladder is retrieved and the marking sutures identifying the transition zone between native trigone and graft were exposed. Cross sections are taken from within the native trigone, the outlined transition zone and the proximally located neo-bladder.

### EXAMPLE 8

### Histological and Immunocytochemical Findings

Specimens are fixed in 10% buffered formalin and processed. Tissue sections are cut at about 4 to about 6 microns for routine staining with Hematoxylin and Eosin (H&E) and Masson's trichrome. Immunocytochemical staining methods are employed with several specific primary antibodies in order to characterize urothelial and smooth muscle cell differentiation in the retrieved bladders. Anti-Desmin antibody (monoclonal NCL-DES-DER11, clone DE-R-11, Novocastra®, Newcastle UK), which reacts with parts of the intermediate filament muscle cell protein desmin, and Anti-Alpha Smooth Muscle Actin antibody (monoclonal NCL-SMA, clone asm-1, Novocastra®, Newcastle UK), which labels bladder smooth muscle actin, are used as general markers for smooth muscle differentiation. Anti-Pancytokeratins AE1/AE3 antibody (monoclonal, Cat. No. 1124 161, Boehringer Mannheim®) and Anti-Cytokeratin 7 antibody (NCL-CK7, Clone LP5K, IgG2b, Novocastra®, New Castle, UK) which react against intermediate filaments that form part of the cytoskeletal complex in epithelial tissues, are used to identify urothelium. Anti-Asymmetric Unit Membrane (AUM) staining, using polyclonal antibodies, is used to investigate the presence of mammalian uroplakins, which form the apical plaques in mammalian urothelium and play an important functional role during advanced stages of urothelial differentiation. Anti S-100 antibody (Sigma®, St. Louis Mo., No. IMMH-9), reacting with the acidic calcium-binding protein S— 100, mainly present in Schwann cells and glial elements in the nervous system, is used to identify neural tissues.

Specimens are fixed in Carnoy's solution or other acceptable fixative for immunohistochemical staining and routinely processed for immunostaining. High temperature antigen unmasking pretreatment with about 0.1% trypsin is performed using a commercially available kit according to the manufacturer's recommendations (Sigma, St. Louis Mo., T-8 128). Antigen-specific primary antibodies are applied to the deparaffinized and hydrate tissue sections. Negative controls are treated with plain serum instead of the primary antibody. Positive controls consist of normal bladder tissue. After washing with phosphate buffered saline, the tissue sections are incubated with a biotinylated secondary antibody and washed again. A peroxidase reagent is added and upon substrate addition, the sites of antibody deposition are visualized by a brown precipitate. Counterstaining is performed with Gill's hematoxylin.

### EXAMPLE 9

### All-in-One Seeding/Bioreactor/Shipping Container

This Example provides a closed system (also referred to herein as an “all-in-one” system) for seeding, growing and shipping neo-organ constructs described herein.

Initial Containers Used from Sterilization to Shipping of Scaffold.

The process of manufacturing a neo-organ cell-scaffold construct involves multiple separate steps, including, among others, shaping the scaffold, sterilizing the scaffold, pre-wetting it, seeding the scaffold with cells, incubating the construct, feeding the construct with construct growth medium, exchanging the medium and shipment to the surgical site. The repeated opening of the container holding the neo-organ and the transfer from one container to another opened the process to contamination and cell damage. Various designs for an all-in-one closed container were iteratively developed in order to reduce and ultimately minimize the likelihood of construct contamination and damage.

Prior to cell seeding, a sterilized scaffold undergoes a pre-wetting procedure. Initially a sterile 500 mL NALGENE® polypropylene jar filled with cell growth medium was used for this procedure. At the time of cell seeding, the scaffold was removed from the pre-wetting container using forceps and is placed in an empty sterile cell-seeding container (see FIGS. 2 and 3, originally designed and manufactured by Tengion, Inc.). This cell-seeding container utilized a plastic three quart container as a seeding vessel and bioreactor for the culture period prior to shipping. The container was wider than it was tall which was useful when seeding the scaffold with cells. The scaffold was not stationary or secured within the seeding container and the laboratory technician was required to hold by hand or with an instrument during the seeding process. The lid of the container could be removed for seeding of the scaffold. The lid could then be closed and sealed and the sealed container could be moved between the biosafety cabinet and incubator in order to change medium and seed cells. Once seeding of the cells had been completed, the container was stored in an incubator but moved to a bio-safety cabinet periodically where the container was opened and closed periodically for adding and exchanging cell growth medium. Once the neo-organ has incubated in the bioreactor for the requisite days, it is transported to the shipping container. In the initial studies described herein, the shipping container was a 1 liter NALGENE® polypropylene jar with a screw cap lid with a Teflon seal (FIG. 6). The NALGENE®(D jar contained an inner plastic basket which supported the neo-bladder during transport (FIG. 7). The neo-bladder could be secured to the inner support basket to prohibit movement during the shipping process (FIG. 8). The inner basket could also be removed at the time of surgery. This enabled the surgical team to remove the neo-organ from the outside container, drain the medium, and then place the sterile neo-organ basket onto the surgical field. This shipping container is an original design and is manufactured by Tengion, Inc. The NALGENE® shipping container was chosen for its size and volume requirements necessary for shipping. During shipping, the container was sealed and two layers of parafilm were wrapped around the edge of the lid to prohibit leakage. The shipping container was labeled and placed in a temperature controlled insulated box, sealed, and shipped (FIG. 9).

First Prototype All-In-One Bioreactor

Since the initial design, described above involved a different container for sterilization, pre-wetting, seeding, and shipping, and involved opening these containers multiple times, the need for a single bioreactor throughout the process became important in order to maintain a controlled environment, foster sterility of the product and avoid construct damage as a result of excessive handling. To solve these problems, a series of new designs were developed to provide a closed system (also referred to herein as an “all-in-one” system) for seeding, growing and shipping neo-organ constructs as further described herein.

In one of its early embodiments of the all-in one design, the closed system included a vessel with a lid that could be tightly secured, e.g., a 1×1 liter polypropylene container with a screw cap lid (e.g., Nalgene® or equivalent) fabricated to include a medium outlet tube. The system also included a septum port for seeding the cells, a pump system, such as the Masterflex L/S Standard Digital Pump system, for controlled delivery of culture media. The system also included three male pipe adaptors. Two male pipe adaptors, e.g., NPT male Teflon pipe adapters with luer ends, were used to connect tubing for automatic medium exchange once the neo-organ construct had been seeded, and one male pipe adapter, e.g., NPT male Teflon pipe adaptor, was used to connect tubing for 95% air/5% CO₂ gas exchange. Female pipe adaptors, e.g., NPT female luer fitting closures, were used to cap the male pipe adaptors. The system also included a 0.2 um PTFE filter (Pall Acro 50 Vent Devices), and support collars, e.g., Teflon coated metal interlocking support collars, to support the scaffold construct.

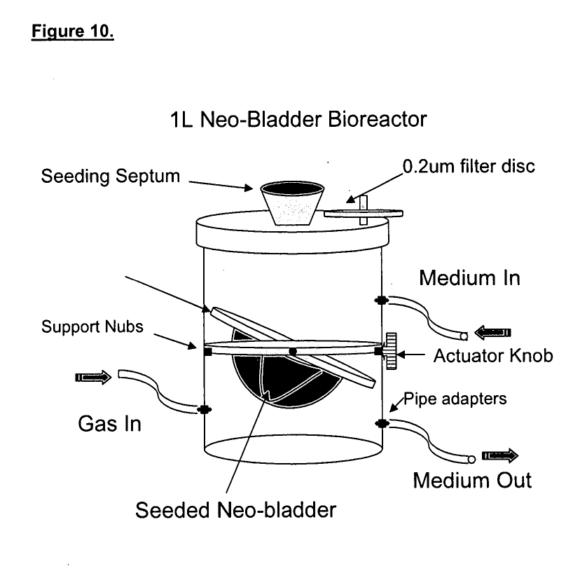

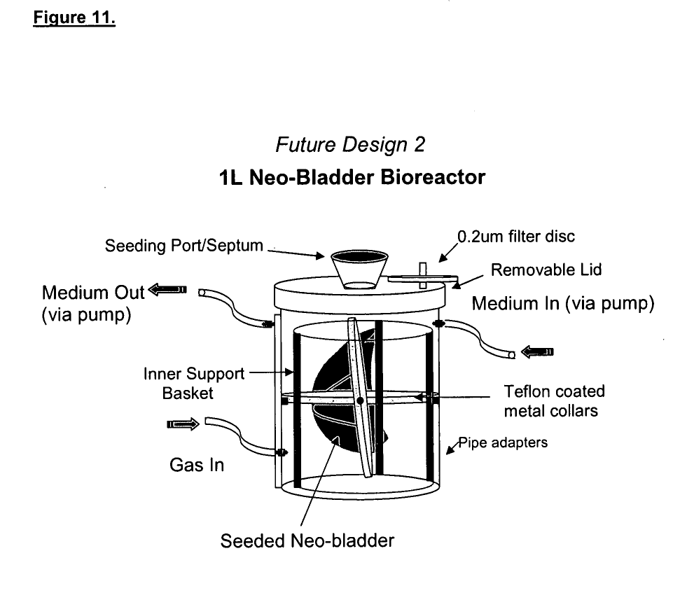

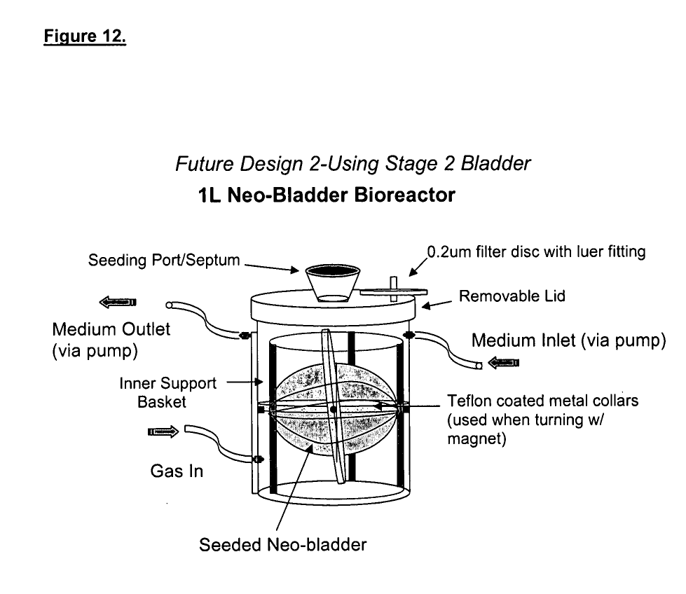

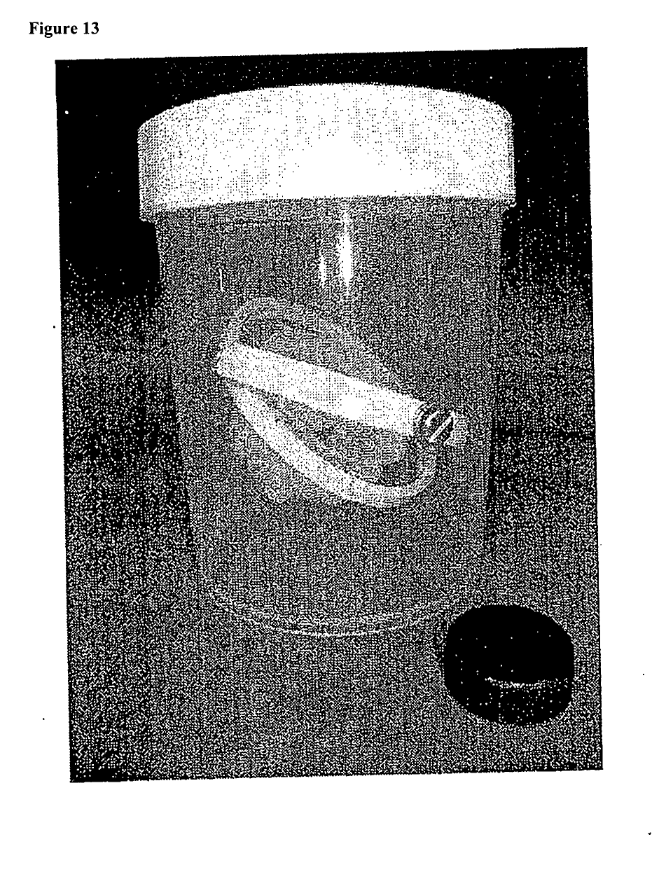

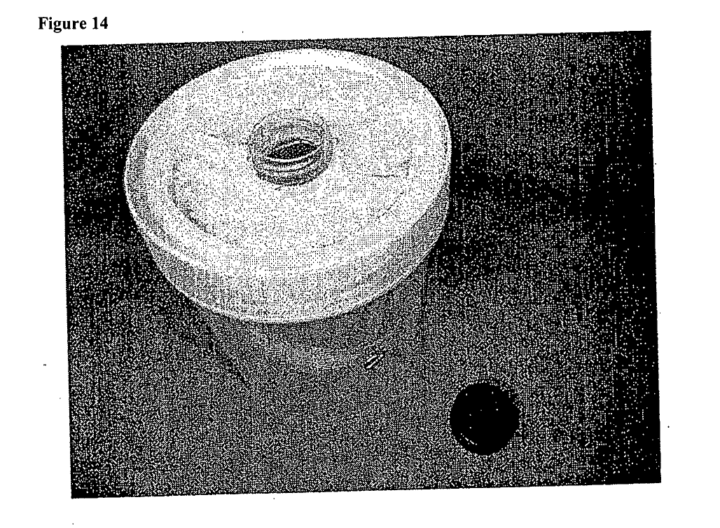

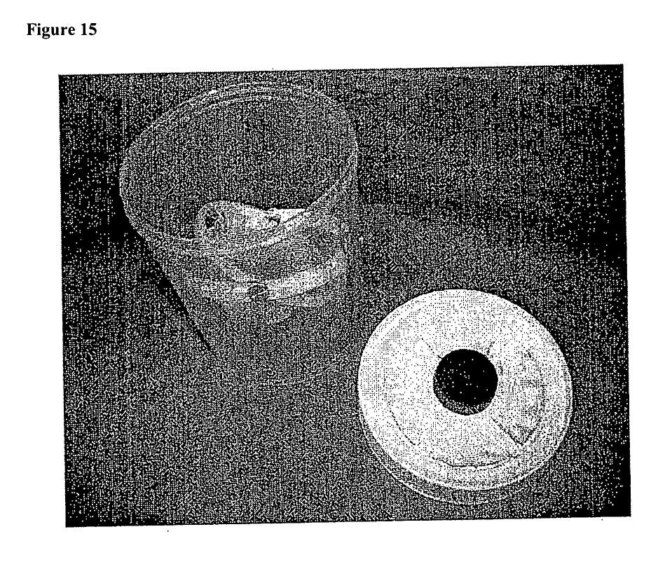

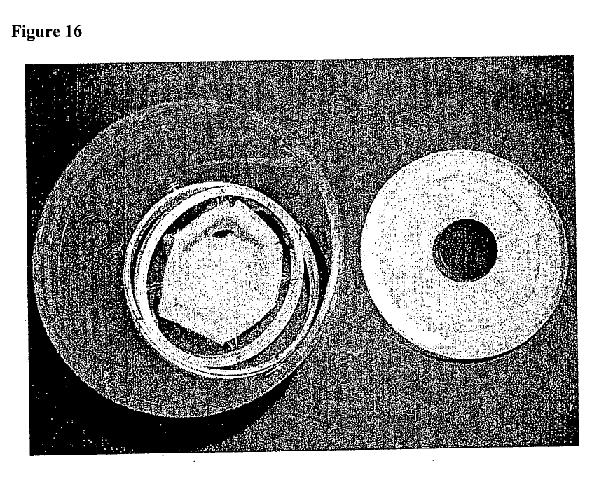

Three specific designs are presented in FIGS. 10-12. In each of these designs, the container could be ethylene oxide sterilized with the coated scaffold inside. This design included two interlocking collars for seeding a secured neobladder scaffold on all surfaces aseptically through a septum port while orienting the scaffold in any required direction. The design also included a gas and medium inlet, as well as a medium outlet, utilizing a pump system to exchange medium in a controlled fashion. All of the ports could be capped and sealed at the time of shipping, to allow for multiple functions. This design had the potential to minimize the manipulation of the scaffold and reduce any possible contamination occurrences due to a more controlled environment. Once the scaffold had been secured in the container, it did not have to be removed until implantation and the technician was not required to directly secure or handle construct. A basic first prototype of this initial “all-in-one” bioreactor is shown in FIGS. 13-16 and features a gyroscope-like ring configuration that allows the scaffold to be positioned for uniform seeding on both sides without direct handling by the technician.

First Engineered Design.

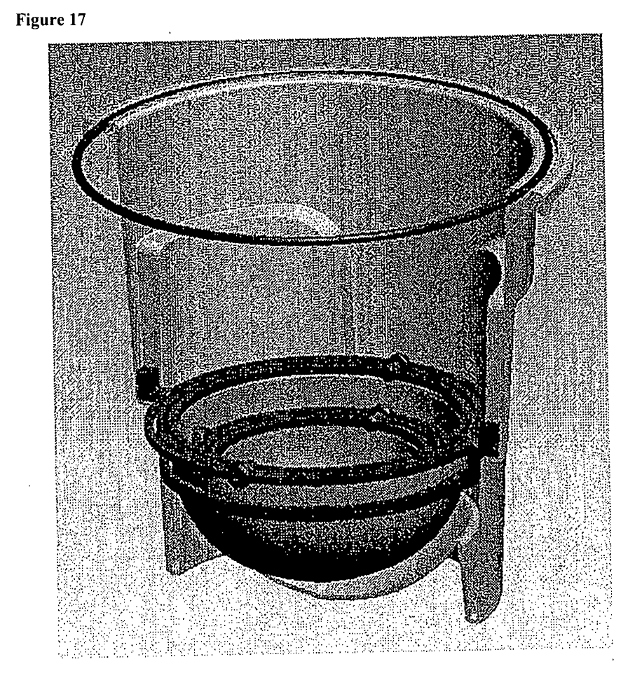

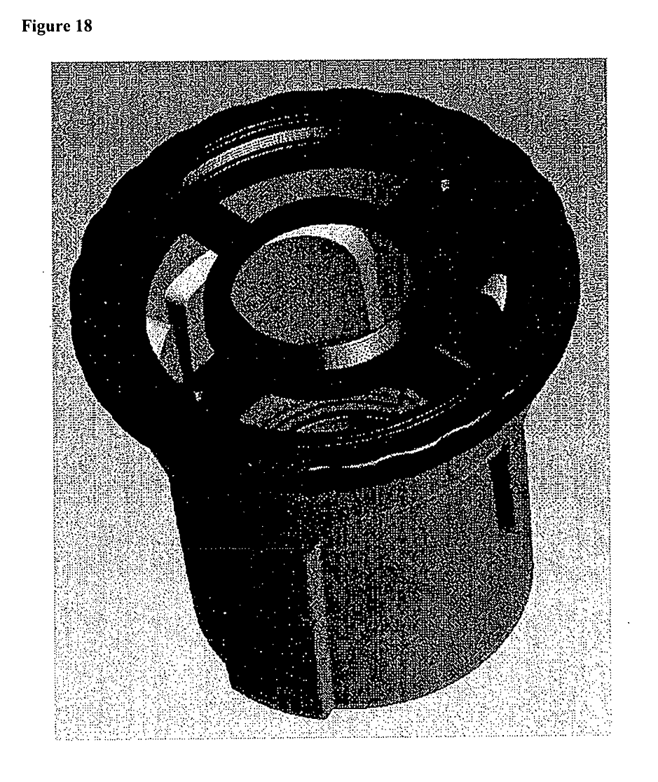

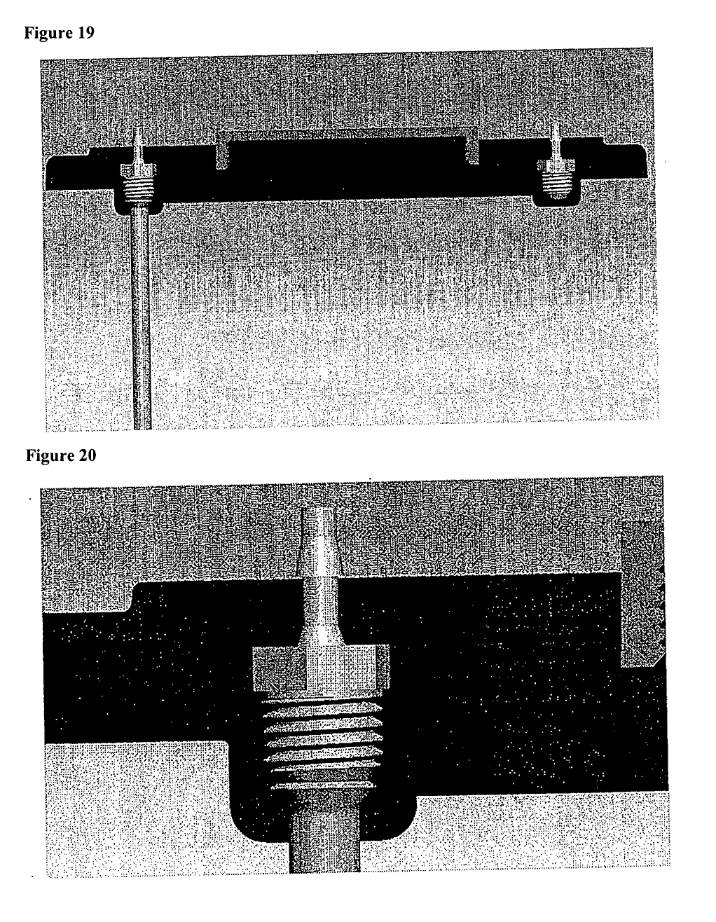

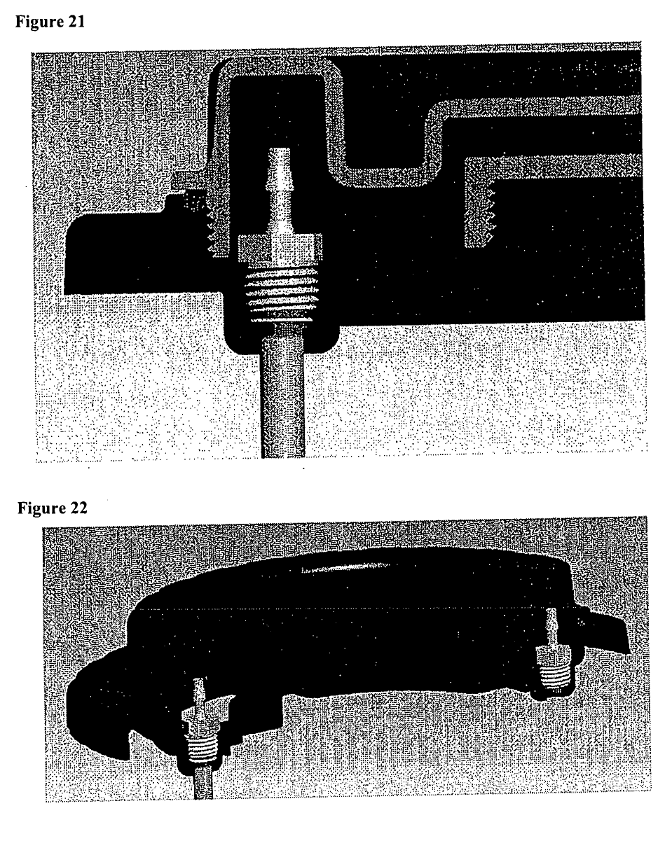

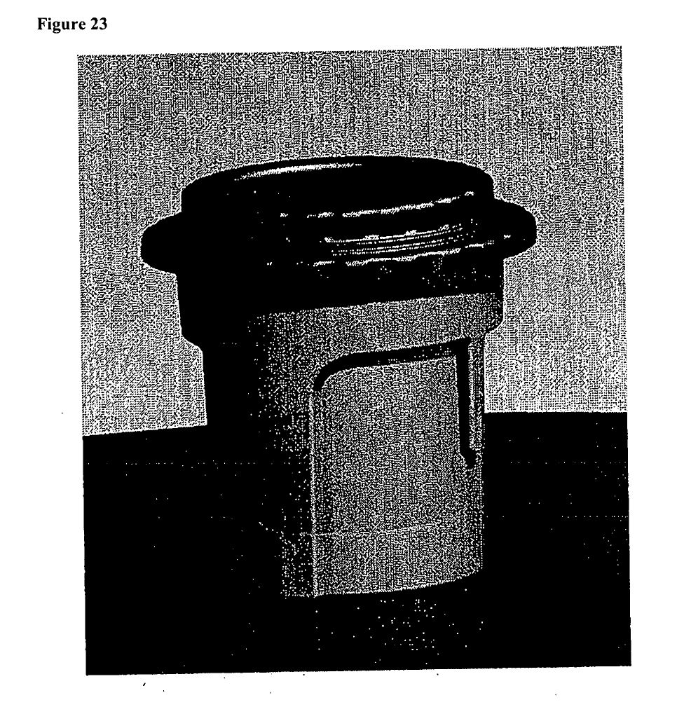

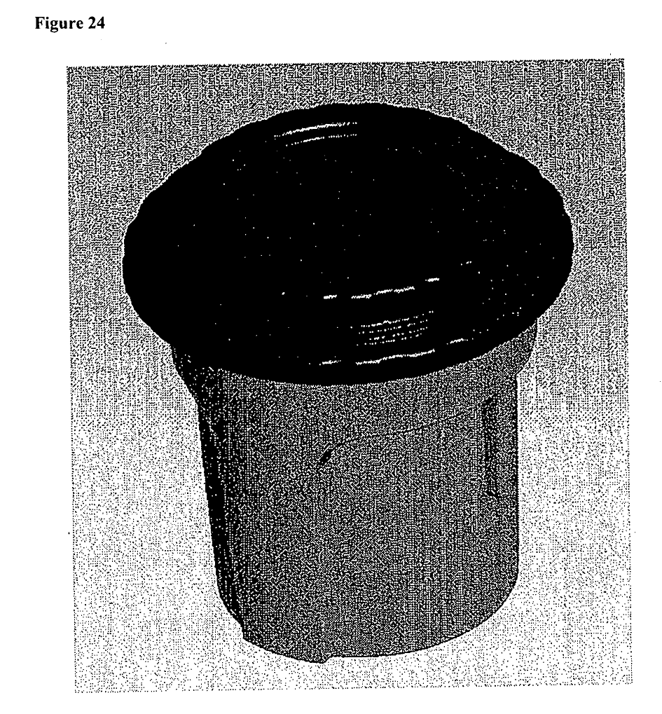

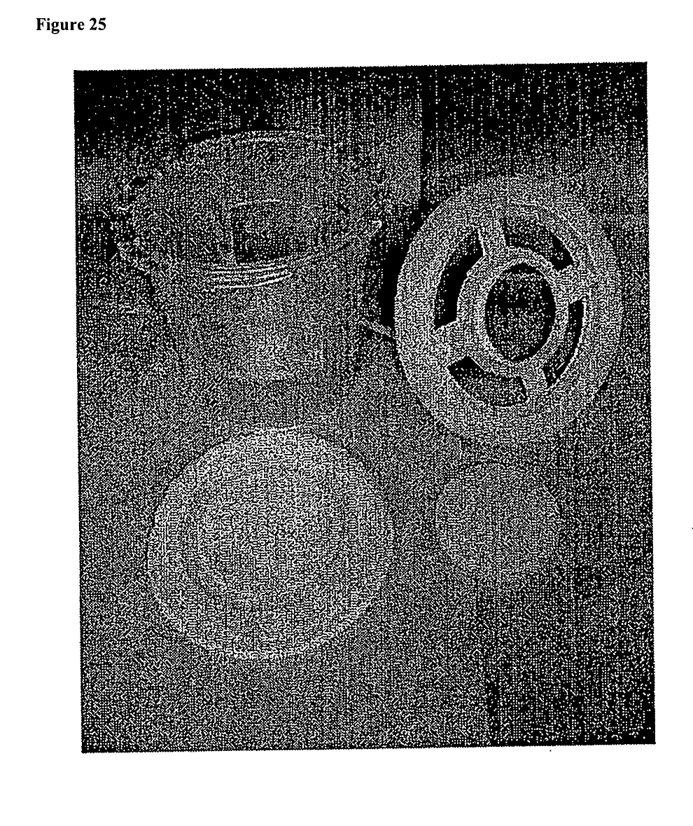

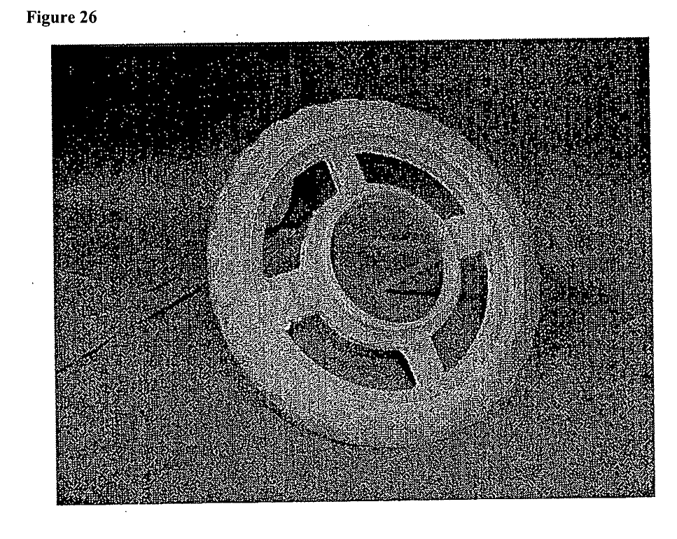

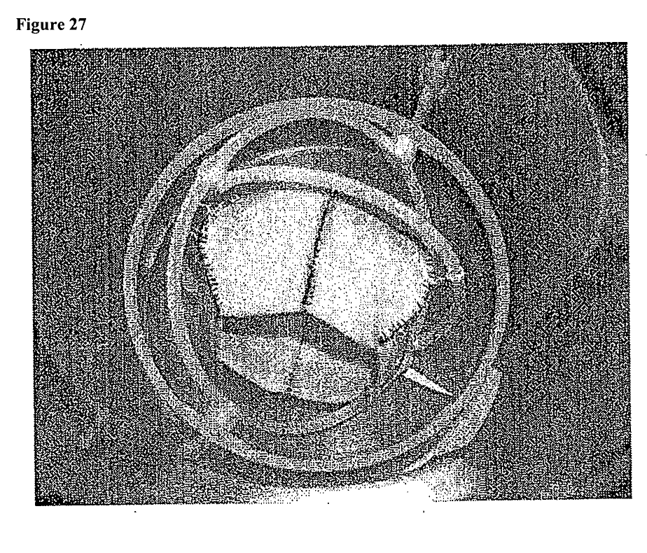

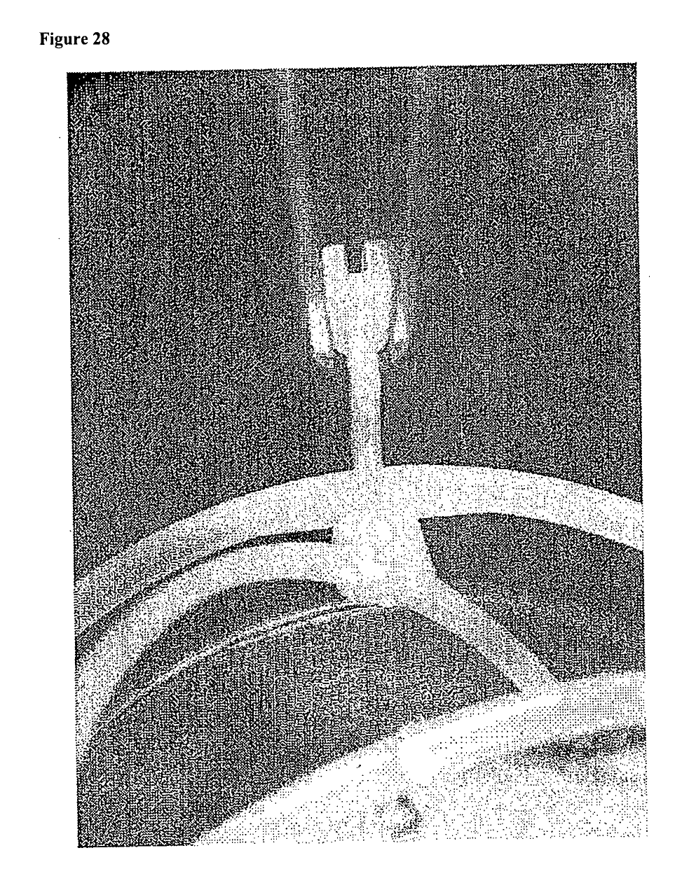

A rapid prototype of the initial “all-in-one” bioreactor design, i.e., a set of drawings and an actual prototype, are shown in FIGS. 17-28. The entire system consists of a container, a lid sealed with an o-ring consisting of 0.2 micron PTFE filter material, a seeding port and culture medium in/out ports with ⅛″ ID tube fittings, 2 culture medium down tubes (flared for sealing purposes when compressed by the tube fittings), a seeding lid, a shipping lid with an o-ring, and four ring assemblies for the 150, 250, 350, and 450 mL scaffold sizes. This design was made into a rapid prototype which was then analyzed for ease of handling and design improvement purposes. Based upon the analysis of this prototype, a second design was implemented before production of the “all-in-one” bioreactor began.

Second Engineered Design.

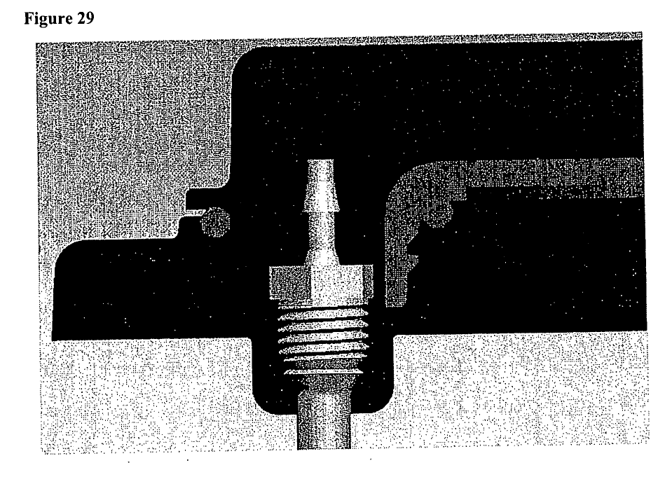

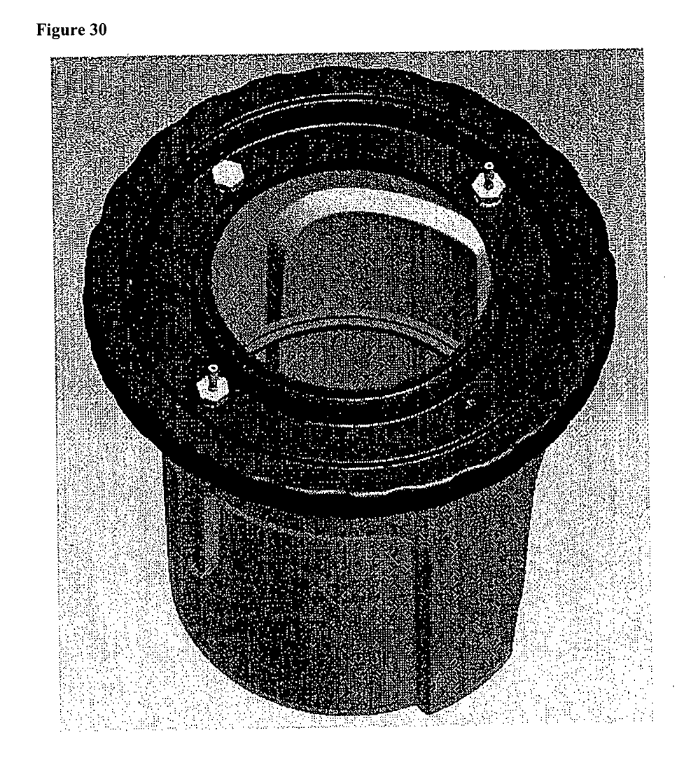

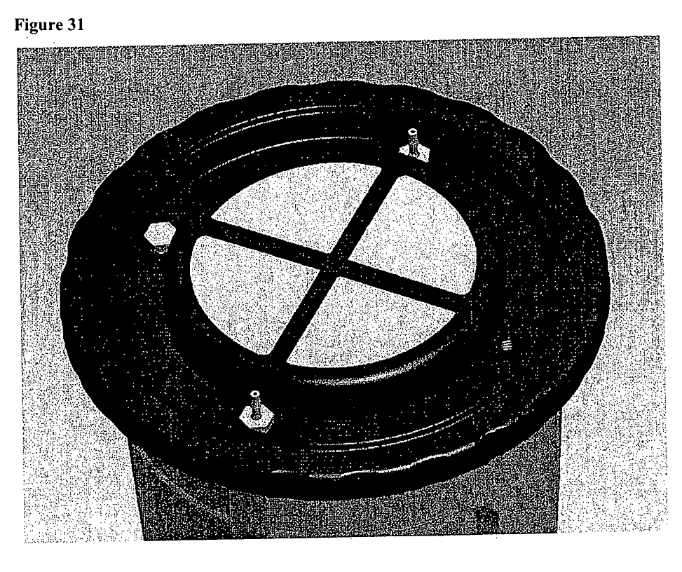

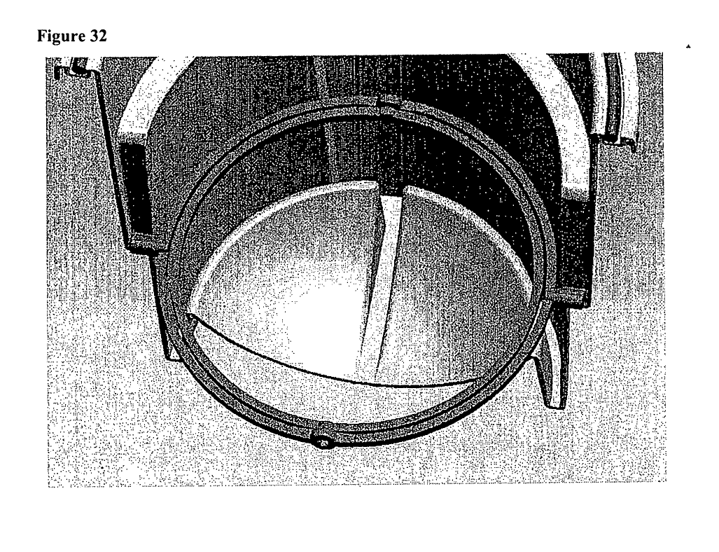

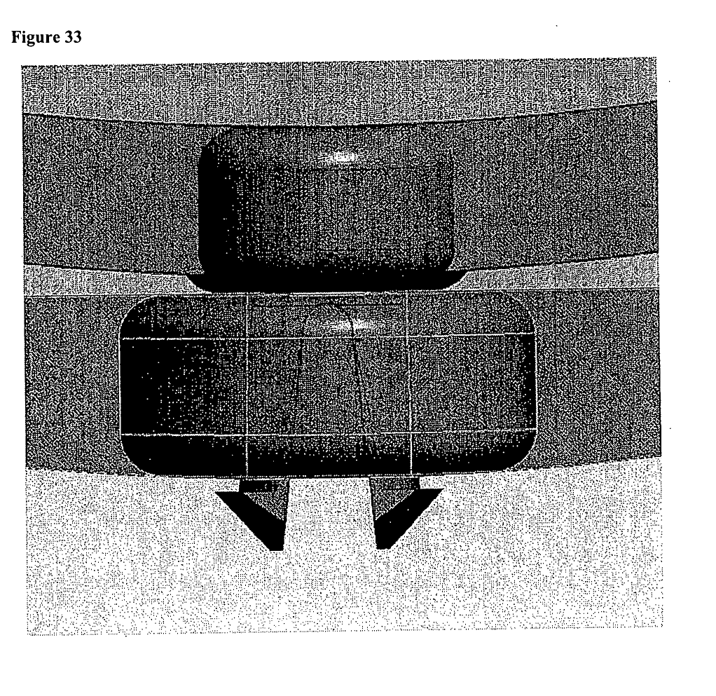

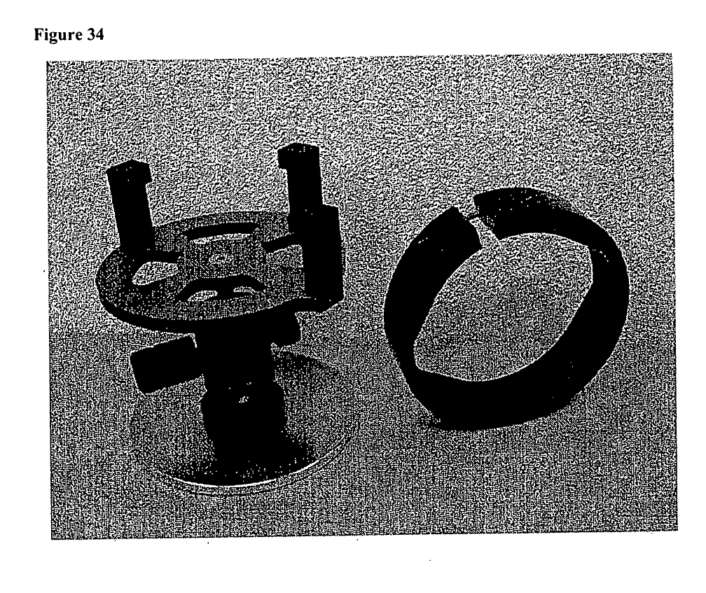

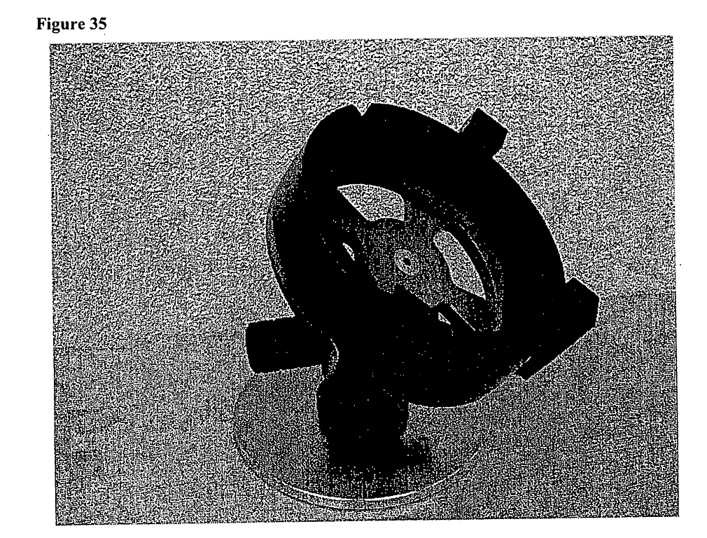

The second design is similar to the first engineered design, with a few improvements and changes made. These changes are reflected in FIGS. 29-33. First, an o-ring seal was added to the seeding port to ensure container closure. Additionally, 2 more ports were added to the main lid for active gas exchange, and the filter material was removed. An option to have filter material on the seeding lid was added, so the seeding lid can be manufactured with filter material if needed. Also, the ring assembly has been designed to fit more tightly to ensure optimal control of the ring movement, which allows for more precise adjustments to the scaffold position when seeding.

Third Engineered Design.

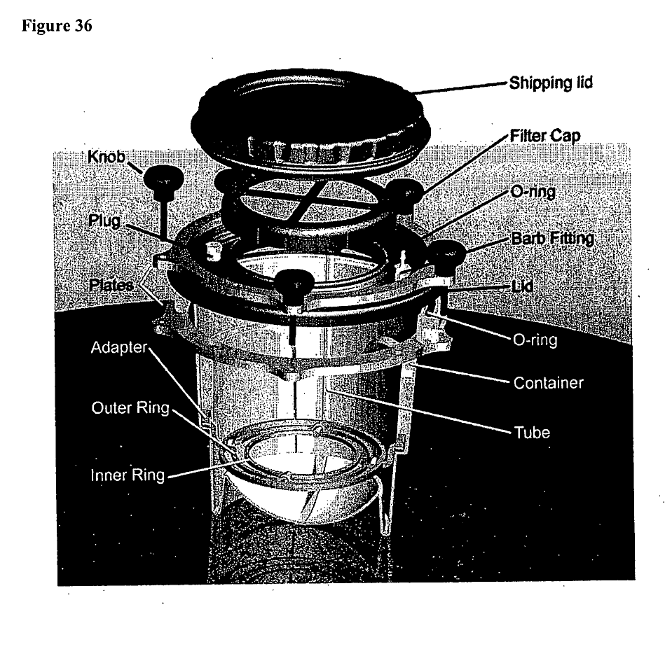

The third design includes alterations made to the main lid to remove the threaded closure and change to a clamp down closure to achieve the seal. The threaded closure could not achieve the necessary clamping force to seal the system under pressure. The clamp ring and knob system addresses this problem and can maintain a seal when under pressure, tested up to 10 psi (internal pressure). See FIG. 36 for a diagram of the clamp rings and knobs.

Additional Equipment.

The main bioreactor container is designed to work with additional equipment. For example, a custom mix of 95% air 5% carbon dioxide with a flowmeter may be used to actively gas the system. Additionally, culture medium bags are utilized to dispense and collect ethanol in the pre-wetting step and to dispense and collect culture medium in the construct growth process via dispensing pump with foot pedal. A stand with ball and socket joint is provided for the bioreactor to achieve an optimal angle for cell seeding of the scaffold. A syringe pump with foot pedal is used to dispense cells onto the construct inside the reactor using a length of tubing and a feeding tube. A tubing welder is used to make sterile welds between tubing attached to the bioreactor and tubing coming from the culture medium bags. A tubing sealer is used to seal at shipping.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim:

1. A closed system bioreactor for producing a neo-organ, said closed system bioreactor comprising:
(a) an outer vessel comprising a securable cap, wherein said securable cap is adapted to mate with an opening in said outer vessel and wherein said securable cap is removable from said opening in said outer vessel;
(b) a medium outlet tube, wherein said medium outlet tube is in fluid communication with the interior of said outer vessel;
(c) a medium inlet tube, wherein said medium inlet tube in fluid communication with the interior of said outer vessel;
(c) a pump system for controlled exchange of medium to and from the interior of said outer vessel, wherein said pump system is coupled to the medium outlet and medium inlet tubes;
(c) a septum port adapted for aseptic access to the interior of said outer vessel;
(d) a gas inlet tube for controlled delivery of a ratio of carbon dioxide and air to the interior of said outer vessel; and
(e) at least one support collar adapted to secure a polymeric matrix shaped to conform to at least a part of a luminal organ or tissue structure within said interior of said outer vessel;

2. The bioreactor of claim 1, wherein said bioreactor comprises at least a first and second support collars, wherein said first and second support collars are interlocking.

3. The bioreactor of claim 2, wherein said interlocking first and second support collars form a gyroscope-like formation in which said first support collar pivots around an axis that is perpendicular to the axis around which the second support collar pivots.

4. The bioreactor of claim 1, wherein said sealable cap comprises a lid and an o-ring that are adapted to mate.

5. The bioreactor of claim 1, wherein said sealable cap is attached to said outer vessel using one or more clamps.

6. The bioreactor of claim 1, wherein said support collar is coated with Teflon.

7. A method for producing a neo-organ construct for the reconstruction, repair, augmentation or replacement of laminarily organized luminal organs or tissue structures in a patient in need of such treatment comprising the steps of:
a) a) providing a biocompatible synthetic or natural polymeric matrix shaped to conform to at least a part of the luminal organ or tissue structure in need of said treatment;
b) sterilizing the matrix at 30 degrees Celsius using ethylene oxide;
c) depositing the first cell population on or in a first area of said polymeric matrix, said first cell population being substantially a muscle cell population;
d) depositing a second cell population of a different cell type than said first cell population in a second area of said polymeric matrix, said second area being substantially separated from said first area; and
e) culturing said first and second cell populations;
wherein steps (b), (c), (d) and (e) occur in a single container.

8. The method of claim 7, wherein the biocompatible material is biodegradable.

9. The method of claim 7, wherein the biocompatible material is polyglycolic acid.

10. The method of claim 7, wherein the second cell population is substantially a urothelial cell population.

11. The method of claim 7, wherein the first cell population is substantially a smooth muscle cell population.

12. The method of claim 7, wherein the luminal organ or tissue structure is of genitourinary organ.

13. The method of claim 7, wherein the luminal organ or tissue structure is selected from the group consisting of bladder, ureters and urethra.

14. The method of claim 13, wherein the luminal organ or tissue structure is a bladder or bladder segment and having urothelial cells deposited on the inner surface of said matrix and smooth muscle cells deposited on the outer surface of said matrix.

15. The method of claim 7, wherein the laminarily organized luminal organ or tissue structure formed in vivo exhibits the compliance of natural bladder tissue.

16. The method of claim 7, wherein said first and second cell populations are deposited sequentially.

17. The method of claim 7, wherein said first and second cell populations are deposited on separate matrix layers and said matrix layers are combined after the deposition steps.

18. The method of claim 7, wherein said single container comprises a closed system.

19. The method of claim 18, wherein said closed system is not physically opened after said first and second cell populations are seeded.
